SEQUENCE LISTING (141 sequences)

SEQ ID NO: 1 - protein, 10 aa, Artificial Sequence
  Synthetic peptide
APAPAPAPAP

SEQ ID NO: 2 - protein, 10 aa, Artificial Sequence
  Synthetic peptide
GGGGSGGGGS

SEQ ID NO: 3 - protein, 10 aa, Artificial Sequence
  Synthetic peptide
EPKSCDKTHT

SEQ ID NO: 4 - protein, 5 aa, Artificial Sequence
  Synthetic peptide
EAAAK

SEQ ID NO: 5 - protein, 10 aa, Artificial Sequence
  Synthetic peptide
EAAAKEAAAK

SEQ ID NO: 6 - protein, 15 aa, Artificial Sequence
  Synthetic peptide
EAAAKEAAAKEAAAK

SEQ ID NO: 7 - protein, 12 aa, Artificial Sequence
  Synthetic peptide
AEAAAKEAAAKA

SEQ ID NO: 8 - protein, 17 aa, Artificial Sequence
  Synthetic peptide
AEAAAKEAAAKEAAAKA

SEQ ID NO: 9 - protein, 7 aa, Artificial Sequence
  Synthetic peptide
HHHHHHH

SEQ ID NO: 10 - protein, 8 aa, Artificial Sequence
  Synthetic peptide
  misc_feature (2)..(2): Xaa can be any naturally occurring amino acid
  misc_feature (5)..(6): Xaa can be any naturally occurring amino acid
CXCCXXCC

SEQ ID NO: 11 - protein, 18 aa, Artificial Sequence
  Synthetic peptide
KDHLIHVHLEEHAHAHNK

SEQ ID NO: 12 - protein, 11 aa, Artificial Sequence
  Synthetic peptide
TMEDYKDDDDK

SEQ ID NO: 13 - protein, 11 aa, Artificial Sequence
  Synthetic peptide
MEQKLISEEDL

SEQ ID NO: 14 - protein, 11 aa, Artificial Sequence
  Synthetic peptide
MASMTGGQQMG

SEQ ID NO: 15 - protein, 15 aa, Artificial Sequence
  Synthetic peptide
KETAAAKFERGHMDS

SEQ ID NO: 16 - protein, 26 aa, Artificial Sequence
  Synthetic peptide
KRRWKKNFIAVSAANRFKKISSSGAL

SEQ ID NO: 17 - protein, 14 aa, Artificial Sequence
  Synthetic peptide
LGIFEAMKMEWRAA

SEQ ID NO: 18 - protein, 10 aa, Artificial Sequence
  Synthetic peptide
SAWRHPQFGG

SEQ ID NO: 19 - protein, 7 aa, Artificial Sequence
  Synthetic peptide
WHPQFEK

SEQ ID NO: 20 - protein, 15 aa, Artificial Sequence
  Synthetic peptide
GLNDIFEAQKIEWHE

SEQ ID NO: 21 - protein, 9 aa, Artificial Sequence
  Synthetic peptide
DVEAWLGAR

SEQ ID NO: 22 - protein, 14 aa, Artificial Sequence
  Synthetic peptide
PTKAKRRVVQREKP

SEQ ID NO: 23 - protein, 14 aa, Artificial Sequence
  Synthetic peptide
GCSGRLICTTNVPW

SEQ ID NO: 24 - protein, 22 aa, Artificial Sequence
  Synthetic peptide
LLSSWGCKGRLVCYTSVQWNET

SEQ ID NO: 25 - protein, 22 aa, Artificial Sequence
  Synthetic peptide
LLSLWGCRGKAVCYTSVQWNET

SEQ ID NO: 26 - protein, 12 aa, Artificial Sequence
  Synthetic peptide
RILAVERYLKDQ

SEQ ID NO: 27 - protein, 16 aa, Artificial Sequence
  Synthetic peptide
RLLGIWGCSGKLICTT

SEQ ID NO: 28 - protein, 28 aa, Artificial Sequence
  Synthetic peptide
RALETLLNQQRLLNSWGCKGRLVCYTSV

SEQ ID NO: 29 - protein, 16 aa, Artificial Sequence
  Synthetic peptide
NTRKSIRIGPGQTFIA

SEQ ID NO: 30 - protein, 14 aa, Artificial Sequence
  Synthetic peptide
RKSVHIGPGQAFYA

SEQ ID NO: 31 - protein, 4 aa, Artificial Sequence
  Synthetic peptide
GGSG

SEQ ID NO: 32 - protein, 6 aa, Artificial Sequence
  Synthetic peptide
GGGASG

SEQ ID NO: 33 - protein, 412 aa, Artificial Sequence
  Synthetic peptide
MGAPTKAKRRVVQREKPHASVIKPEMKIKLRMEGAVNGHKFVIEGEGIGKPYEGTQTLDLTVEEGCSGRLICTTNVPWGGSGAPLPFSYDILTPAFQYGNRAFTKYPEDIPDYFKQAFPEGYSWERSMTYEDLLSSWGCKGRLVCYTSVQWNETQGICIATSDITMEGGSGLLSLWGCRGKAVCYTSVQWNETDCFFYEIRFDGTNFRILAVERYLKDQKKTLKWEPSTEKMYVEDRLLGIWGCSGKLICTTGGSGVLKGDVEMALLLEGGRALETLLNQQRLLNSWGCKGRLVCYTSVGHYRCDFKTTYKAKKDNTRKSIRIGPGQTFIAAHEVDHRIEILSHDKRLNSWGCKGRLVCYTSVDYNKVRLYEHAEARYSGGGSGGGASGRKSVHIGPGQAFYAHHHHHHKKK

SEQ ID NO: 34 - protein, 406 aa, Artificial Sequence
  Synthetic peptide
GAPTKAKRRVVQREKPHASVIKPEMKIKLRMEGAVNGHKFVIEGEGIGKPYEGTQTLDLTVEEGCSGRLICTTNVPWGGSGAPLPFSYDILTPAFQYGNRAFTKYPEDIPDYFKQAFPEGYSWERSMTYEDLLSSWGCKGRLVCYTSVQWNETQGICIATSDITMEGGSGLLSLWGCRGKAVCYTSVQWNETDCFFYEIRFDGTNFRILAVERYLKDQKKTLKWEPSTEKMYVEDRLLGIWGCSGKLICTTGGSGVLKGDVEMALLLEGGRALETLLNQQRLLNSWGCKGRLVCYTSVGHYRCDFKTTYKAKKDNTRKSIRIGPGQTFIAAHEVDHRIEILSHDKRLNSWGCKGRLVCYTSVDYNKVRLYEHAEARYSGGSGARKSVHIGPGQAFYAHHHHHHKKK

SEQ ID NO: 35 - DNA, 1221 bp, Artificial Sequence
  Synthetic primer
atgggagcccccacgaaggcaaagcgtcgtgttgtccaacgtgagaagccacatgcgagcgttatcaagcctgaaatgaaaattaaactgcgcatggagggggcagtaaatgggcacaagtttgtcattgagggggaagggattggcaaaccttacgaagggacccagactctggacttgacggtggaggaggggtgcagtggccgccttatctgtaccacgaatgtcccatggggtggctctggcgccccccttcctttctcctacgatattcttacgccggcgtttcagtacgggaaccgtgctttcaccaagtacccagaggatattccagactactttaagcaggctttccccgaggggtactcatgggagcgttcaatgacgtacgaggatctgcttagtagctggggttgcaaagggcgtcttgtttgctacacctccgtgcaatggaacgagacgcaagggatttgtattgctaccagtgacatcacaatggaaggggggtccgggttgttatctttgtgggggtgccgtggcaaggcagtatgctatacgagtgtacagtggaatgaaacagattgctttttttatgaaatccgctttgatggtacaaatttccgtatcttggcggttgaacgctatttgaaggatcagaagaagactttgaaatgggagccatccactgaaaaaatgtacgtagaggatcgcttgttaggaatttggggctgtagtggcaaattgatttgcactactggcggaagcggtgtccttaagggtgacgtagaaatggccttacttttagaaggcggccgcgctttagaaaccttgcttaatcagcagcgccttctgaattcatgggggtgtaaaggacgcttggtgtgttatacgtccgtgggccattatcgctgcgattttaagactacgtacaaggcaaaaaaggataatacccgcaaaagcattcgcatcgggcctggacagactttcatcgcagcacacgaagtggaccatcgtattgaaattttgtcacacgacaagcgcctgaattcctggggttgtaaaggccgcttggtgtgttatacctcggtggattataacaaggtgcgcttatacgagcacgctgaggcccgttactcaggaggatcgggggcacgtaaatccgtacatatcggtccgggccaagccttttacgctcatcatcaccatcaccataaaaagaag

SEQ ID NO: 36 - protein, 20 aa, Artificial Sequence
  Synthetic peptide
GGWYSFDSPYLMSITEMRLR

SEQ ID NO: 37 - protein, 12 aa, Artificial Sequence
  Synthetic peptide
FWRGDLVFDFQV

SEQ ID NO: 38 - protein, 15 aa, Artificial Sequence
  Synthetic peptide
FNASDSVGQQIKVIP

SEQ ID NO: 39 - protein, 23 aa, Artificial Sequence
  Synthetic peptide
CWGELMNLATWVGSNLEDPASRE

SEQ ID NO: 40 - protein, 9 aa, Artificial Sequence
  Synthetic peptide
ASRDLVVNY

SEQ ID NO: 41 - protein, 12 aa, Artificial Sequence
  Synthetic peptide
ANLINEFDDLAS

SEQ ID NO: 42 - protein, 51 aa, Artificial Sequence
  Synthetic peptide
GGVYLLPRRGPRLGVWHPRGSRPSWGPTDPRRWHNHVSPTHYVPESDAAAR

SEQ ID NO: 43 - protein, 20 aa, Artificial Sequence
  Synthetic peptide
FFTEEFKRKNKGKNLASSHR

SEQ ID NO: 44 - protein, 21 aa, Artificial Sequence
  Synthetic peptide
RFFVQGDGIGQHSLQEALERR

SEQ ID NO: 45 - protein, 18 aa, Artificial Sequence
  Synthetic peptide
SASDQPTQYEEEMTDYQK

SEQ ID NO: 46 - protein, 12 aa, Artificial Sequence
  Synthetic peptide
VKEKEEVKEKEE

SEQ ID NO: 47 - protein, 19 aa, Artificial Sequence
  Synthetic peptide
FAELLEQQKNAQFPGKASK

SEQ ID NO: 48 - protein, 10 aa, Artificial Sequence
  Synthetic peptide
AEPKPAEPKS

SEQ ID NO: 49 - protein, 11 aa, Artificial Sequence
  Synthetic peptide
PPSGTENKPAT

SEQ ID NO: 50 - protein, 15 aa, Artificial Sequence
  Synthetic peptide
KIEFSKFTVKIKNKD

SEQ ID NO: 51 - protein, 41 aa, Artificial Sequence
  Synthetic peptide
MTLFLFISCNNSGKDGNTSABIPSKENAKLIVYFYDNVYAG

SEQ ID NO: 52 - protein, 14 aa, Artificial Sequence
  Synthetic peptide
LRKVGDSVKAASKE

SEQ ID NO: 53 - protein, 19 aa, Artificial Sequence
  Synthetic peptide
ASGAKEEAEKKAAEQRALL

SEQ ID NO: 54 - protein, 16 aa, Artificial Sequence
  Synthetic peptide
TSGKDIVQFAKAVEIS

SEQ ID NO: 55 - protein, 7 aa, Artificial Sequence
  Synthetic peptide
NQSTVKT

SEQ ID NO: 56 - protein, 9 aa, Artificial Sequence
  Synthetic peptide
VLAEAIGLR

SEQ ID NO: 57 - skipped

SEQ ID NO: 58 - protein, 15 aa, Artificial Sequence
  Synthetic peptide
LQPFPQPELPYPQPQ

SEQ ID NO: 59 - protein, 9 aa, Artificial Sequence
  Synthetic peptide
GPKPGAPFP

SEQ ID NO: 60 - protein, 13 aa, Artificial Sequence
  Synthetic peptide
AQYKALPVVLENA

SEQ ID NO: 61 - protein, 12 aa, Artificial Sequence
  Synthetic peptide
TLLRAVESYLLA

SEQ ID NO: 62 - protein, 19 aa, Artificial Sequence
  Synthetic peptide
YTDSSMAVTLMKFASNFLF

SEQ ID NO: 63 - protein, 15 aa, Artificial Sequence
  Synthetic peptide
TVSTEQNVPDPQVGI

SEQ ID NO: 64 - protein, 12 aa, Artificial Sequence
  Synthetic peptide
SHSQLGPPVGPP

SEQ ID NO: 65 - protein, 12 aa, Artificial Sequence
  Synthetic peptide
CLTFGRETVLEY

SEQ ID NO: 66 - protein, 12 aa, Artificial Sequence
  Synthetic peptide
REVLYREFDEME

SEQ ID NO: 67 - protein, 15 aa, Artificial Sequence
  Synthetic peptide
ETWKKPDYEPPVVHG

SEQ ID NO: 68 - protein, 12 aa, Artificial Sequence
  Synthetic peptide
RRVAVLVLLDRL

SEQ ID NO: 69 - protein, 15 aa, Artificial Sequence
  Synthetic peptide
NEIELSARDVLENIG

SEQ ID NO: 70 - protein, 15 aa, Artificial Sequence
  Synthetic peptide
KSTYLTEPILTEEHL

SEQ ID NO: 71 - protein, 7 aa, Artificial Sequence
  Synthetic peptide
KAAAAPA

SEQ ID NO: 72 - protein, 10 aa, Artificial Sequence
  Synthetic peptide
AEPKSAEPKP

SEQ ID NO: 73 - protein, 12 aa, Artificial Sequence
  Synthetic peptide
DSSAHSTPSTPA

SEQ ID NO: 74 - protein, 17 aa, Artificial Sequence
  Synthetic peptide
MKKNDQIVAAIALRGVA

SEQ ID NO: 75 - protein, 15 aa, Artificial Sequence
  Synthetic peptide
LVACSIGLVERTNAA

SEQ ID NO: 76 - protein, 6 aa, Artificial Sequence
  Synthetic peptide
AESPKK

SEQ ID NO: 77 - protein, 7 aa, Artificial Sequence
  Synthetic peptide
VMYASSG

SEQ ID NO: 78 - protein, 26 aa, Artificial Sequence
  Synthetic peptide
KDGKSWKLESHKFDWNTPDPRIGFKD

SEQ ID NO: 79 - protein, 9 aa, Artificial Sequence
  Synthetic peptide
VLKTDVNKE

SEQ ID NO: 80 - protein, 12 aa, Artificial Sequence
  Synthetic peptide
SANNDAEIGNLI

SEQ ID NO: 81 - skipped

SEQ ID NO: 82 - protein, 14 aa, Artificial Sequence
  Synthetic peptide
DAKMTEEDKENALS

SEQ ID NO: 83 - protein, 17 aa, Artificial Sequence
  Synthetic peptide
EICPAVKRDVDLFLTGT

SEQ ID NO: 84 - protein, 12 aa, Artificial Sequence
  Synthetic peptide
EVDHTNFKYNYS

SEQ ID NO: 85 - protein, 19 aa, Artificial Sequence
  Synthetic peptide
DNYWSFSDSTYWTLRYSSG

SEQ ID NO: 86 - protein, 15 aa, Artificial Sequence
  Synthetic peptide
LEVGKQRLKYAQEEL

SEQ ID NO: 87 - protein, 15 aa, Artificial Sequence
  Synthetic peptide
SHSVTKSLRVFGGPP

SEQ ID NO: 88 - protein, 6 aa, Artificial Sequence
  Synthetic peptide
TPPAYR

SEQ ID NO: 89 - protein, 15 aa, Artificial Sequence
  Synthetic peptide
KTKRNTNRRPQDVKF

SEQ ID NO: 90 - protein, 20 aa, Artificial Sequence
  Synthetic peptide
AFASRGNHVSPTHYVPESDA

SEQ ID NO: 91 - protein, 15 aa, Artificial Sequence
  Synthetic peptide
SVPTNLDYVPQFLRW

SEQ ID NO: 92 - protein, 10 aa, Artificial Sequence
  Synthetic peptide
PEPTVTNEEY

SEQ ID NO: 93 - protein, 7 aa, Artificial Sequence
  Synthetic peptide
KAAIAPA

SEQ ID NO: 94 - protein, 15 aa, Artificial Sequence
  Synthetic peptide
GTSEEGSRGGSSMPS

SEQ ID NO: 95 - protein, 12 aa, Artificial Sequence
  Synthetic peptide
FGQAAAGDKPSL

SEQ ID NO: 96 - protein, 15 aa, Artificial Sequence
  Synthetic peptide
DTGSERSIRYRRRVY

SEQ ID NO: 97 - protein, 14 aa, Artificial Sequence
  Synthetic peptide
SQVADKDDPTNKFY

SEQ ID NO: 98 - protein, 10 aa, Artificial Sequence
  Synthetic peptide
PVVAESPKKP

SEQ ID NO: 99 - protein, 7 aa, Artificial Sequence
  Synthetic peptide
DYARVMY

SEQ ID NO: 100 - protein, 28 aa, Artificial Sequence
  Synthetic peptide
ETKAWYPYLKDGKSWKLESHKFDWNTPD

SEQ ID NO: 101 - protein, 10 aa, Artificial Sequence
  Synthetic peptide
TKDASIDYHE

SEQ ID NO: 102 - protein, 12 aa, Artificial Sequence
  Synthetic peptide
GDBRKAMFEDIA

SEQ ID NO: 103 - skipped

SEQ ID NO: 104 - protein, 20 aa, Artificial Sequence
  Synthetic peptide
ELISSCLVWSARGCLFGGGI

SEQ ID NO: 105 - protein, 14 aa, Artificial Sequence
  Synthetic peptide
EVLPPPRKKKGLFS

SEQ ID NO: 106 - protein, 12 aa, Artificial Sequence
  Synthetic peptide
PPNAPILSTLPE

SEQ ID NO: 107 - protein, 15 aa, Artificial Sequence
  Synthetic peptide
RKTSERSQPRGRRQP

SEQ ID NO: 108 - protein, 15 aa, Artificial Sequence
  Synthetic peptide
SVPTNLDYVPQYLRW

SEQ ID NO: 109 - protein, 14 aa, Artificial Sequence
  Synthetic peptide
GFDDGSAFGGGLPF

SEQ ID NO: 110 - protein, 15 aa, Artificial Sequence
  Synthetic peptide
GDKPSPFGQAAAADK

SEQ ID NO: 111 - protein, 11 aa, Artificial Sequence
  Synthetic peptide
SPFGQAAAGDK

SEQ ID NO: 112 - protein, 9 aa, Artificial Sequence
  Synthetic peptide
CNNSGKDGN

SEQ ID NO: 113 - protein, 20 aa, Artificial Sequence
  Synthetic peptide
INKLEAKKTSLKTYSEYEEQ

SEQ ID NO: 114 - protein, 14 aa, Artificial Sequence
  Synthetic peptide
KDDPTNKFYQSVIQ

SEQ ID NO: 115 - protein, 13 aa, Artificial Sequence
  Synthetic peptide
VQEGVQQEGAQQP

SEQ ID NO: 116 - protein, 7 aa, Artificial Sequence
  Synthetic peptide
EAAFREL

SEQ ID NO: 117 - protein, 20 aa, Artificial Sequence
  Synthetic peptide
LEIGYERFKTKGIRDSGSKE

SEQ ID NO: 118 - protein, 10 aa, Artificial Sequence
  Synthetic peptide
TRLIDERAAH

SEQ ID NO: 119 - protein, 12 aa, Artificial Sequence
  Synthetic peptide
SAPLKQIAANAG

SEQ ID NO: 120 - protein, 21 aa, Artificial Sequence
  Synthetic peptide
AGAGGGAGGAGAGGGAGGAGC

SEQ ID NO: 121 - protein, 12 aa, Artificial Sequence
  Synthetic peptide
GLLRYHTYARFG

SEQ ID NO: 122 - protein, 8 aa, Artificial Sequence
  Synthetic peptide
EALESPEH

SEQ ID NO: 123 - protein, 15 aa, Artificial Sequence
  Synthetic peptide
QKKNKRNTNRRPQDV

SEQ ID NO: 124 - protein, 10 aa, Artificial Sequence
  Synthetic peptide
IVEVEEILPE

SEQ ID NO: 125 - protein, 10 aa, Artificial Sequence
  Synthetic peptide
DEMLDPEASF

SEQ ID NO: 126 - protein, 9 aa, Artificial Sequence
  Synthetic peptide
KQKAAEATK

SEQ ID NO: 127 - protein, 14 aa, Artificial Sequence
  Synthetic peptide
CVQEGVQQEGAQQP

SEQ ID NO: 128 - protein, 14 aa, Artificial Sequence
  Synthetic peptide
SDISSTTGKPDSTG

SEQ ID NO: 129 - protein, 8 aa, Artificial Sequence
  Synthetic peptide
TTNRFAKT

SEQ ID NO: 130 - protein, 8 aa, Artificial Sequence
  Synthetic peptide
TLNPTIAG

SEQ ID NO: 131 - protein, 12 aa, Artificial Sequence
  Synthetic peptide
NAGKEGAIIFQQ

SEQ ID NO: 132 - protein, 12 aa, Artificial Sequence
  Synthetic peptide
FWRGDLVFDFQV

SEQ ID NO: 133 - protein, 14 aa, Artificial Sequence
  Synthetic peptide
FGRETVLEYLVSFG

SEQ ID NO: 134 - protein, 18 aa, Artificial Sequence
  Synthetic peptide
PPLLESWKDPDYVPPWHG

SEQ ID NO: 135 - protein, 13 aa, Artificial Sequence
  Synthetic peptide
KNEEFLNDRCDIC

SEQ ID NO: 136 - protein, 8 aa, Artificial Sequence
  Synthetic peptide
DPNANPNV

SEQ ID NO: 137 - protein, 6 aa, Artificial Sequence
  Synthetic peptide
PVVAES

SEQ ID NO: 138 - protein, 5 aa, Artificial Sequence
  Synthetic peptide
VETLL

SEQ ID NO: 139 - protein, 15 aa, Artificial Sequence
  Synthetic peptide
AKKAILITDAAKDKG

SEQ ID NO: 140 - protein, 12 aa, Artificial Sequence
  Synthetic peptide
SANNDAEIGNLI

SEQ ID NO: 141 - protein, 12 aa, Artificial Sequence
  Synthetic peptide
GAIIFQQVMSRS

(19) United States
(12) Patent Application Publication
(10) Pub. No.: US 2022/0205991 A1
(43) Pub. Date: Jun. 30, 2022

(54) Controlled Generation of Measurable Signals and Uses Thereof

(71) Applicant: Retala Leasing, LLC, The Colony, TX (US)

(72) Inventors: Ross Teggatz, The Colony, TX (US); Aaron Frank, Murphy, TX (US)

(21) Appl. No.: 17/432,845

(22) PCT Filed: Feb. 21, 2020
(86) PCT No.: PCT/US2020/019158

§ 371 Date: Aug. 20, 2021

Related U.S. Application Data
(60) Provisional application No. 62/809,361, filed on Feb. 22, 2019.

(51) Int. Cl. G01N 33/543; B01L 3/00; G01N 27/327

(52) U.S. Cl. CPC G01N 33/5438; B01L 3/502; G01N 27/3273; B01L 2200/026; B01L 2200/16; B01L 2300/0645; B01L 2300/0663; B01L 2300/123; B01L 2300/161; B01L 2200/0689

(57) ABSTRACT

The present invention includes a device and method comprising: one or more wells on a substrate onto which one or more molecules of interest (MOI) binding reagents are attached, wherein each of the one or more MOI binding reagents is within a molecular proximity of one or more detectable signal molecules, wherein each of the one or more detectable signal molecules comprise one or more signal molecules that are releasable in the presence of the MOI by one or more enzymes and the signal is detected by an electronic detection system.

### CROSS-REFERENCE TO RELATED APPLICATIONS

None.

### STATEMENT OF FEDERALLY FUNDED RESEARCH

None.

### TECHNICAL FIELD OF THE INVENTION

The present invention relates in general to the field of the controlled generation of measureable signals and uses thereof.

### BACKGROUND OF THE INVENTION

Without limiting the scope of the invention, its background is described in connection with the detection of molecules of interest.

The generation of a signal dependent on the presence of an analyte/molecule of interest in a test sample is a common laboratory procedure and a multitude of methods have been developed. In many instances, the process either detects antibodies or relies on the binding capabilities of antibodies to perform the analysis. However, no one system exists that allows for the rapid detection of indicators or agents of disease into quantifiable signals that shows high portability and robust performance in non-laboratory spaces without the need for specialized training to operate and conduct the analysis.

Conventional laboratory-based testing relies on large centralized equipment to perform analyses on large volume blood samples that are collected through venipuncture by a phlebotomist or other trained technicians. In many instances, the patient sample is collected at a site distantly separated from the centralized equipment location, which requires the storage and transport of the sample. In addition to the possibility of corrupting the quality of the sample, there is a time component that can extend to hours and days or even weeks for extremely remote collection locations. However, the major disadvantages include a requirement for skilled sample collection, sample storage and transport, and the time to obtain results.

Lateral flow tests most commonly utilized a chromatic detection system for positive test results (presence of the MOI) that can be ambiguous and often requires that the untrained end user determine the results, which can lead to human error in the final result. The chromatic system also limits the sensitivity of a lateral flow test due to the need to meet level of human visual activity. Lastly, the chromatic system has a narrow temporal window for providing valid results. The format of the system, which uses capillary action within an absorbent matrix to allow interactions between the MOI and the anchored binding reagent limits the number of independent MOIs that can be measured in a single test. To perform a test, the lateral flow system requires that the user apply multiple different solutions in series, which again is an opportunity for human error to influence the results.

Nucleic acid-based tests represent another class of analyses for detection of MOIs, more specifically pathogens. This approach requires a biological sample that contains the pathogen, which can greatly limit its effectiveness for pathogens that display a short temporal window for presence in the biological system. Extensive protocols and the professional collection of biological samples are required, which can restrict its use outside of medical/laboratory facilities. Currently, large expensive equipment is needed to perform the analysis. Another manner to use the genomic DNA of a pathogen for detection and identification is Polymerase Chain Reaction that can amplify a specific region of pathogen DNA defined by the sequences in two, single stranded oligonucleotides. A major disadvantage is that the system is easily contaminated, and internal controls are not possible.

Other lab-on-the chip systems have been developed that are mostly miniaturized versions of lab-based systems that have not yet been realized. Surface Plasmon Resonance (SPR) systems have the potential to allow label-free detection of MOI. Yet the systems are large and non-mobile as well as being expensive, and further, they require a highly trained technician and extensive calibration and maintenance of the equipment.

All of these approaches are highly dependent on immunological reagents for the capture of the MOI and secondary reagents to detect that event (ie capture of the MOI). Current approaches to provide those reagents are inefficient and suffer from long development times since they cannot be designed in silico and depend on the natural systems of various animals as well as humans. Furthermore, each final product is unique that requires the empirical determination of the best method for its production which is a long, involved and expensive endeavor. Overall, existing technologies have limited flexibility that often do not deliver the level of selectivity and specificity needed for highly confident results.

First generation immunological reagents typically consist of preparations of biologically active pathogens (virus, bacteria, parasite). While these reagents provide high sensitivity because they include all possible epitopes, the specificity of the reagent is low due to the cross-reactivity to closely related pathogens. Furthermore, many of the processes were potential biohazards that required costly containment systems (Biosafety Level—BSL II-IV).

FIG. 1 one approach of the prior art which is the transition to semi-purified proteins or recombinant production of proteins from a pathogen could increase specificity, yet still showed cross-reactivity and lower than optimal specificity. Furthermore, the sensitivity of the system is reduced since there are fewer classes of antibodies that can be bound. Recombinant proteins can be used, but multiple proteins are needed to represent a pathogen that increases the production costs. Since individual proteins can have multiple epitopes, the potential for cross reactivity is not mitigated.

FIG. 2 shows another approach of the prior art which is use of the most specific element of a pathogen for detection by antibodies are the short amino acid sequences of between 5-14 amino acids in the antigenic proteins of the pathogen, epitopes. Importantly, epitopes that are removed from a whole protein environment and represented in a peptide format normally has a lower performance profile in terms of binding antibodies compared to the same sequence in a whole protein. In the peptide form, the affinity for its corresponding antibody is frequently less when the same peptide sequence is present within a whole protein.

FIG. 3 shows another approach of the prior art which is the inclusion of epitopes related to the of medically relevant in a subunit Chimeric/Fusion recombinant protein can present the epitope, but due to the fact that there are other epitope strands attached to the protein similar antibodies with IgG associated with the epitope strands can cause binding. This means that a variety of similar antibody strains can potentially attach to a given fusion protein, not allowing for specific association.

Manufacturing time for all the above approaches are extensive and expensive. Difficulties of performing QC using other non-fluorescent core proteins. Representative sample from the lot.

Thus, despite these many advances a need remains for a robust, easy to use, predictable, and easy to maintain system and method for the detection of molecules of interest from samples (biological, environmental, etc.), which system can be deployed under difficult conditions and operated by individuals with little medical training

### SUMMARY OF THE INVENTION

In one embodiment, the present invention includes a device comprising: one or more wells on a substrate onto which one or more molecules of interest (MOI) binding reagents are attached, wherein each of the one or more MOI binding reagents is within a molecular proximity of one or more detectable signal molecules, wherein each of the one or more detectable signal molecules comprise one or more signal molecules that are releasable in the presence of the MOI by one or more enzymes. In one aspect, a release of the one or more signal molecules is measured electronically to determine a quantity of the one or more MOIs in the sample. In another aspect, the device comprises a cartridge that comprises the substrate, the wells, and the MOI, in fluid communication with one or more ports 34-36 that facilitate the transfer of chemical reagents into and out of the wells. In another aspect, the device further comprises a pump in fluid communication with the ports, which the pump is external to the cartridge. In another aspect, the cartridge comprises a body constructed of plastic, and wherein the cartridge is machining, injection molding, or 3D printing. In another aspect, the device does not require valves. In another aspect, a diameter of one or more fluid pathways is hydrophobic. In another aspect, the cartridge the cartridge includes one or more circuits in electrical communication with the substrate and the wells, wherein the one or more circuit convert electrochemical measurements into electrical signals.

In another embodiment, the present invention includes a valveless device for detecting a molecule of interest (MOI) comprising: one or more reagents in a liquid; a pump in fluid communication with the reagents and a reaction chamber, wherein the reaction chamber comprises one or more substrates onto which one or more MOI binding reagents are attached, wherein each of the one or more MOI binding reagents is within a molecular proximity of one or more detectable signal molecules; a port for introducing a sample suspected of comprising the MOI into the reaction chamber; wherein each of the one or more detectable signal molecules comprise one or more signal molecules that are releasable in the presence of the MOI by one or more enzymes; and wherein a release of the one or more signal molecules is measured to determine a quantity of the one or more MOIs in the sample; and one or more sensors capable of detecting the one or more signal molecules released if the MOI is present in the sample. In another aspect, the liquid and pump are contained within a cartridge. In another aspect, the liquid, pump, port, reaction chamber, and sensors are sealed within a closed system. In another aspect, the one or more reagents are contained within a disposable bag or capsule. In another aspect, the port comprises an elastomeric seal that prevent liquid from escaping the one or more conduits within the device. In another aspect, the substrate is a planar surface. In another aspect, the one or more sensors are defined further as capture molecule functionalized electrodes.

Also taught herein is a reaction-based method for the rapid detection of one or more molecules of interest (MOIs) in a sample comprising: providing one or more MOI binding reagents, wherein each of the one or more MOI binding reagents is within a molecular proximity of one or more detectable signal molecules; wherein each of the one or more detectable signal molecules comprise one or more signal molecules that are releasable in the presence of the MOI by one or more enzymes; and

wherein a release of the one or more signal molecules is measured electronically to determine a quantity of the one or more MOIs in the sample. In one aspect, the method further comprises attaching each of the one or more MOI binding reagents a signal detection surface, wherein each of the one or more MOI binding reagents is attached to a unique, known region of the signal detection surface, wherein binding of the one or more MOIs to a specific MOI binding reagent triggers an electronic signal at the unique, known region of the signal detection surface that indicates that the MOI bound to the MOI binding reagent specific for that MOI. In another aspect, the method further comprises providing one or more fluidic channels in fluid contact between the sample and the one or more MOI binding reagents. In another aspect, an electronic signal detected is at least one of: non-transient, cumulative, or coded. In another aspect, the MOI is selected from pathogen-specific antibodies, auto-antibodies, viruses, bacteria, parasites, fungi, helminths, chemicals, illicit drugs, drugs, toxins, hormones, proteins, lipids, glycogens, carbohydrates, biological therapies, pathogen biomarkers, cardiac biomarkers, disease biomarkers, or cancer biomarkers. In another aspect, the samples are selected from at least one of biological fluids, water, air and surfaces. In another aspect, the further comprising MOI binding reagent is an engineered beta-barrel chimeric protein comprising one or more polypeptides that specifically interact with the MOI, antibodies, an antibody fragment, Fab, Fab', Fab'-SH, F(ab')2, Fv, or scFv fragment, nanobody, a bi-specific antibody, pathogen lysate, recombinant protein, chimeric proteins peptide, glycopeptide, lectin or carbohydrate. In another aspect, the one or more MOIs comprise a negative control for a presence or absence of the MOI, a positive control for the presence or absence of the MOI, both a positive and a negative control for the presence or absence of the MOI. In another aspect, the reaction-based method occurs at a signal detection surface, wherein the surface comprises the one or more regions that each comprise a different MOI binding reagent and detect a different MOI, wherein the MOIs further comprise at least one of: (1) a positive control for the presence of a sample; (2) a positive control for the presence of an activating enzyme; (3) a positive control for the detection of a signal; or (4) a negative control that does not generate any signal. In another aspect, the one or more regions of the signal detection surface detects a different MOI by having a different MOI binding reagent at that surface, different regions of the same MOI, or one or more epitopes of a pathogen, virus, bacteria, parasite, fungi, helminth, chemical, illicit drug, drug, toxin, hormone, protein, lipid, glycogen, carbohydrate, biological therapy, pathogen biomarker, cardiac biomarker, disease biomarker, or cancer biomarker.

In another aspect, the one or more MOI binding reagents, one or more detectable signal, and one or more enzymatic reactions, are incorporated into a cartridge in fluid communication with an electronic surface capable of detecting the one or more signal molecules and the one or more validation signals. In another aspect, a signal measured electronically are solid state detectors. In another aspect, the enzymatic reaction occurs in one or more closed system cartridges. In another aspect, the electronically measured signal are an electrochemical, a surface plasmon resonance, an infrared, a capacitance coupled, a dye-coupled fiber optic, a hyperspectral sensor or a cantilever sensor. In another aspect, the method further comprises an internal control that is at least one of: an internal calibration for signal intensity, signal production kinetics, or signal position. In another aspect, the MOI is selected from an antibody, an antibody fragment, Fab, Fab', Fab'-SH, F(ab')2, Fv, or scFv fragment, nanobody, or a bi-specific antibody. In another aspect, the one or more MOI binding agents are a primary binding agent, and a second MOI binding agent localizes to the molecular proximity of the primary binding agent that depends on the presence of the MOI. In another aspect, the one or more detectable signal molecules comprises a single nucleic acid molecules that comprises multiple regions, wherein at least a first region physically attaches to a specific molecular space with the MOI binding reagent, a second region doubles back to form a double stranded segment that can be acted upon by a specific enzyme dependent on the presence of the MOI, and a segment region connected to the signal molecule that is released by an enzyme. In another aspect, the one or more detectable signal molecules are a first nucleic acid, and further comprising adding a second nucleic acid at least partially complementary to the first nucleic acid, wherein at least one of the first and second nucleic acids comprises a detectable signal, wherein the detectable signal is release from the at least one of the first and second nucleic acids in the presence of an enzyme that cuts the first and second nucleic acids when double stranded. In another aspect, the second nucleic acid is conjugated to one or more enzymes that release the signal attached to the first nucleic acid. In another aspect, the enzymatic reaction comprises one or more enzymes that are specific for a same double stranded nucleic acid target sequence, to a different nucleic acid target sequences, or are specific for multiple nucleic acid target sequences. In another aspect, the enzyme is selected from at least one of a DNA methylase, methyl-dependent restriction enzyme, a heterodimeric restriction enzyme, an asymmetric restriction enzyme or a DNA nicking enzyme. In another aspect, the enzyme is selected from at least one of FokI, Mva 19691, BtsI, BbvCI, BfiI, BsrDI, BstNBI/BspD6I, Dam/DpnI, Fsp 4H1/Bis I, AluI/AluI. In another aspect, the MOI binding agent is not detected by an enzyme-linked immunosorbent assay (ELISA), radioimmunoassay, luminescence assay, fluorescent microscopy, microarray or fluorescence-activated cell sorting (FACS) analysis. In another aspect, the signal molecule is coded to link a signal measurement to the identity of the MOI. In another aspect, the electronic detection system comprises multiple independent electrodes that allows a subset to include: an internal calibration for signal intensity, signal production kinetics, and a signal position. In another aspect, the method further comprises a second MOI binging protein conjugated to, or in a fusion protein with, an enzyme capable of functioning within the molecular proximity of the one or more detectable signal molecules and that acts to release the signal molecule. In another aspect, the method further comprises a second MOI binding protein is an antibody, an antibody fragment, Fab, Fab', Fab'-SH, F(ab')2, Fv, or scFv fragment, nanobody, a beta-barrel fusion protein comprising at least one exogenous polypeptide, or a bi-specific antibody. In another aspect, the method further comprises a second detectable signal molecule that comprises a second signal molecule, wherein each of a first and the second signal molecules are measured, and a ratio between the first and the second signal molecules measures a relative amount of a first and a second MOI.

Also taught herein is a molecule of interest (MOI) binding reagent comprising an engineered beta-barrel fusion protein comprising one or more exogenous polypeptide sequences inserted at three or more locations between two adjacent beta strand sequences of a beta-barrel protein, and wherein the three or more exogenous polypeptide sequences are epitopes that are ligands or substrates for the MOI. In another aspect, the one or more exogenous polypeptide sequences are selected from linear or three-dimensional peptides from a proteome of viruses, eukaryotes, or prokaryotes. In another aspect, the protein is selected from a neurotrophic factor; ribosomal protein L14; alcohol dehydrogenase; asparty1-tRNA synthetase; staphylococcal nuclease mutant; lipoamide dehydrogenase; transferase; thrombin; DMSO reductase; elongation factor; acidic fibroblast growth factor; TATA-box binding protein; aconitase; pyruvate kinase; oxidoreductase (superoxide acceptor); triose phosphate isomerase; enolase; retinol-binding protein; retinoic acid binding protein; green fluorescent protein; alpha-hemolysin; porin; maltoporin; GTP cyclohydrolase I, mCerulean, DsRed, mcavFP, YFP, eGFP, eBFP, asFP, zoanRFP, zsGFP, zYFP538, mCherry, mcavRFP, or mcavGFP. In another aspect, the protein comprises 4, 5, 6, 7, 8, 11, 14, 16, 18, or 20 beta strands that form a barrel. In another aspect, the e MOI is an antibody, an enzyme, a receptor, a ligand, a DNA binding protein, a ligand, a substrate, signaling protein, a lectin, pathogen, virus, bacteria, parasite, fungi, helminth, chemical, illicit drug, drug, toxin, hormone, protein, lipid, glycogen, carbohydrate, biological therapy, pathogen biomarker, cardiac biomarker, disease biomarker, or cancer biomarker. In another aspect, the method further comprises a second detectable signal molecule that comprises a second signal molecule, wherein each of a first and the second signal molecules are measured, and a ratio between the first and the second signal molecules measures a relative amount of a first and a second MOI.

Also taught herein is a method for identifying an epitope of a molecule of interest (MOI) comprising: expressing an engineered beta-barrel fusion protein that comprises the one or more epitopes of a molecule of interest (MOI), wherein the epitopes are inserted at one or more locations between two beta stands sequences of a beta-barrel protein; and contacting the one or more antibodies to the engineered beta-barrel fusion protein, wherein antibodies that bind to the one or more epitopes of the engineered beta-barrel fusion protein are epitopes for the antibodies. In another aspect, the method further comprises removing from a sample comprising one or more antibodies to a beta-barrel protein that does not comprises any epitopes of the MOI to remove antibodies that bind to the beta-barrel protein. In another aspect, the express in a cell and isolate the bacteria. In another aspect, at least one of the epitopes are a membrane insertion tag, a membrane localization tag, a maltose binding protein tag, a polyhistidine tag, a flag tag, a myc tag, an influenza hemagglutinin (HA) tag, a glutathione-s-transferase (GST) tag, a nus tag, a CD20 tag, Her2/neu tag, T7-tag, an S tag, a GFP tag, an Avi-Tag, a calmodulin-binding peptide, a streptavidin-binding peptide, a chitin-binding domain, or a SNAP-tag.

Also taught herein is a nucleic acid comprising an engineered beta-barrel fusion protein that comprises three or more exogenous polypeptide sequences comprising epitopes of a molecule of interest (MOI), wherein the epitopes are inserted at one or more locations between two beta stands sequences of a beta-barrel protein. In another aspect, the nucleic acid is expressed in a cell and the engineered beta-barrel fusion protein is secreted or bound to a surface of the cell.

Also taught herein is a method for the rapid detection of one or more antibodies that specifically bind to an epitope of a molecule of interest (MOI) comprising: obtaining one or more antibodies raised against the epitopes of the MOI; contacting the one or more antibodies to an engineered beta-barrel fusion protein that comprises the one or more epitopes of the MOI, wherein the epitopes are inserted at one or more locations between two beta stands sequences of a beta-barrel protein; and detecting which of the one or more antibodies bind to the one or more epitopes of the engineered beta-barrel fusion protein. In another aspect, the method further comprises the step of isolating the antibody. In another aspect, the method further comprises the step of purifying the antibody.

Also taught herein is a system for the rapid detection of one or more molecules of interest (MOIs) in a sample comprising: one or more regions of a surface to detect an MOI; one or more MOI binding reagents, wherein each of the one or more MOI binding reagents is within a molecular proximity of one or more detectable signal molecules that comprise at least a first nucleic acid sequence; wherein each of the one or more detectable signal molecules comprise one or more signal molecules that are releasable in the presence of the MOI by one or more enzymes; and wherein a release of the one or more signal molecules is measurable electronically to determine a quantity of the one or more MOIs in the sample. In another aspect, the MOI is selected from pathogen-specific antibodies, auto-antibodies, viruses, bacteria, parasites, fungi, helminths, chemicals, illicit drugs, drugs, toxins, hormones, proteins, lipids, glycogens, carbohydrates, biological therapies, pathogen biomarkers, cardiac biomarkers, disease biomarkers, or cancer biomarkers. In another aspect, the samples are selected from at least one of biological fluids, water, air and surfaces. In another aspect, the MOI binding reagent is an engineered beta-barrel chimeric protein comprising one or more polypeptides that specifically interact with the MOI, antibodies, from an antibody, an antibody fragment, Fab, Fab', Fab'-SH, F(ab')2, Fv, or scFv fragment, nanobody, a bi-specific antibody, pathogen lysate, recombinant protein, chimeric proteins peptide, glycopeptide, lectin or carbohydrate. In another aspect, the one or more MOIs comprise a negative control for a presence or absence of the MOI, a positive control for the presence or absence of the MOI, both a positive and a negative control for the presence or absence of the MOI. In another aspect, the enzymatic reaction occurs on a surface, wherein the surface comprises the one or more regions that each detect a different MOI, wherein the MOIs are selected from at least one of: (1) a positive control for the presence of a sample; (2) a positive control for the presence of an activating enzyme; (3) a positive control for the detection of a signal; or (4) a negative control that does not generate any signal. In another aspect, the one or more regions of the surface detects a different MOI, different regions of the same MOI, different epitopes of an MOI, different pathogens, viruses, bacteria, parasites, fungi, helminths, chemicals, illicit drugs, drugs, toxins, hormones, proteins, lipids, glycogens, carbohydrates, biological therapies, pathogen biomarkers, cardiac biomarkers, disease biomarkers, or cancer biomarkers. In another aspect, the one or more MOI binding reagents, one or more detectable signal, and one or more enzymatic reactions, are incorporated into a cartridge in fluid communication with an electronic surface capable of detecting the one or more signal molecules and the one or more validation signals. In another aspect, a signal measured electronically are solid state detectors. In another aspect, the enzymatic reaction occurs in one or more closed system cartridges. In another aspect, the electronically measured signal are an electrochemical, a surface plasmon resonance, an infrared, a capacitance coupled, a dye-coupled fiber optic or a hyperspectral sensor or a cantilever sensor. In another aspect, the system further comprises an internal control that is at least one of: an internal calibration for signal intensity, signal production kinetics, or signal position. In another aspect, the MOI is selected from an antibody, an antibody fragment, Fab, Fab', Fab'-SH, F(ab')₂, Fv, or scFv fragment, nanobody, or a bi-specific antibody. In another aspect, the one or more MOI binding agents are a primary binding agent, and a second MOI binding agent localizes to the molecular proximity of the primary binding agent that depends on the presence of the MOI. In another aspect, the one or more detectable signal molecules comprises a single nucleic acid molecules that comprises multiple regions, wherein at least a first region physically attaches to a specific molecular space with the MOI binding reagent, a second region doubles back to form a double stranded segment that can be acted upon by a specific enzyme dependent on the presence of the MOI, and a segment region connected to the signal molecule that is released by an enzyme. In another aspect, the one or more detectable signal molecules are a first nucleic acid, and further comprising adding a second nucleic acid at least partially complementary to the first nucleic acid, wherein at least one of the first and second nucleic acids comprises a detectable signal, wherein the detectable signal is release from the at least one of the first and second nucleic acids in the presence of an enzyme that cuts the first and second nucleic acids when double stranded. In another aspect, the second nucleic acid is conjugated to one or more enzymes that release the signal attached to the first nucleic acid. In another aspect, the enzymatic reaction comprises one or more enzymes that are specific for a same double stranded nucleic acid target sequence, to a different nucleic acid target sequences, or are specific for multiple nucleic acid target sequences. In another aspect, the enzyme is selected from at least one of a DNA methylase, methyl-dependent restriction enzyme, a heterodimeric restriction enzyme, an asymmetric restriction enzyme or a DNA nicking enzyme. In another aspect, the enzyme is selected from at least one of FokI, Mva 19691, BtsI, BbvCI, BfiI, BsrDI, BstNBI/BspD6I, Dam/DpnI, Fsp 4H1/Bis I, AluI/AluI. In another aspect, the MOI binding agent is not detected by an enzyme-linked immunosorbent assay (ELISA), radioimmunoassay, luminescence assay, fluorescent microscopy, microarray or fluorescence-activated cell sorting (FACS) analysis. In another aspect, the signal molecule is non-transient. In another aspect, the signal molecule is coded to link a signal measurement to the identity of the MOI. In another aspect, the electronic detection system comprises multiple independent electrodes that allows a subset to include: an internal calibration for signal intensity, signal production kinetics, and a signal position. In another aspect, the method further comprises a second detectable signal molecule that comprises a second signal molecule, wherein each of a first and the second signal molecules are measured, and a ratio between the first and the second signal molecules measures a relative amount of a first and a second MOI.

### BRIEF DESCRIPTION OF THE DRAWINGS

For a more complete understanding of the features and advantages of the present invention, reference is now made to the detailed description of the invention along with the accompanying figures and in which:

FIG. 1 shows one approach of the prior art, which is the transition to semi-purified proteins or recombinant production of proteins from a pathogen.

FIG. 2 shows another approach of the prior art which is use of the most specific element of a pathogen for detection by antibodies are the short amino acid sequences of between 5-14 amino acids in the antigenic proteins of the pathogen, epitopes.

FIG. 3 shows another approach of the prior art which is the inclusion of epitopes related to medically relevant in a subunit Chimeric/Fusion recombinant protein can present the epitope.

FIG. 4 is an illustration of MOI-A bound to an MRC with binding agent A′ while the MRC with binding agent B′ does not have MOI-B.

FIG. 5 is an illustration of a unique biochemical reagent that includes a section that can bind a region common to all MOIs and a separate section capable of performing a reaction on a chemical compound shown in FIG. 1.

FIG. 6 is an illustration of the unique biochemical reagent in FIG. 4 bound to MOI-A on the surface of the MRC. No reagent is bound to the MRC with binding agent B′.

FIG. 7 is an illustration of a section of the unique biochemical reagent interacting with the chemical compound associated with the binding agent A′ on the surface of the MRC.

FIG. 8 is an illustration of the result of the unique biochemical reagent interacting with the chemical compound associated with the binding agent A′ on the surface of the MRC such that there is a release of a portion of the chemical compound.

FIG. 9 is an illustration of two electrodes each with a unique chemical compound A′″ and B′″ that can interact specifically with portion of the chemical compound released by the actions of the unique biochemical reagent.

FIG. 10 is a representative flowchart of one embodiment of the present invention.

FIG. 11 shows a high-level block diagram of one embodiment of the present invention.

FIGS. 12A, 12B, and 12C show, respectively, a cross-sectional side view, a cross-sectional top view, and an external view of a device for use with the present invention.

FIG. 13A shows a top view of a device circuitry, and 13B a cross-section of a sensor for use with the present invention.

FIG. 14 is an illustration of the combination of a reaction reagent with its corresponding 1st chemical compound on the surface of a solid substance.

FIG. 15 shows that the natural sequence of a protein can be replicated for recombinant protein production or the sequence can be codon optimized for the production host for recombinant protein production.

FIG. 16 shows the alignment of multiple fluorescent proteins from Aequorea Victoria and Discosoma sp, Montastraea cavernosa, Zoanthus sp and Montastraea c avernosa. The overall structural elements of the eleven β-sheets are easily detected. It is also clear that the absolute amino acid sequence is not maintained over the course of evolution. The intervening sequences between the β-sheets also show divergence, which allows them to serve as insertion points for epitopes.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “antigen” refers to a molecule that can initiate a humoral and/or cellular immune response in a recipient of the antigen. Antigens include any type of biologic molecule, including, for example, simple intermediary metabolites, sugars, lipids and hormones as well as macromolecules such as complex carbohydrates, phospholipids, nucleic acids and proteins. Common categories of antigens include, but are not limited to, viral antigens, bacterial antigens, fungal antigens, protozoan and other parasitic antigens, tumor antigens, antigens involved in autoimmune disease, allergy and graft rejection, and other miscellaneous antigens.

As used herein, the term “epitope(s)” refer to a peptide or protein antigen that includes a primary, secondary or tertiary structure similar to an epitope located within any of a number of pathogen polypeptides encoded by the pathogen DNA or RNA. The level of similarity will generally be to such a degree that monoclonal or polyclonal antibodies directed against such polypeptides will also bind to, react with, or otherwise recognize, the peptide or protein antigen. Various immunoassay methods may be employed in conjunction with such antibodies, such as, for example, Western blotting, ELISA, RIA, and the like, all of which are known to those of skill in the art. The identification of pathogen epitopes, and/or their functional equivalents, suitable for use in vaccines is part of the present invention. Once isolated and identified, one may readily obtain functional equivalents. For example, one may employ the methods of Hopp, as taught in U.S. Pat. No. 4,554,101, incorporated herein by reference, which teaches the identification and preparation of epitopes from amino acid sequences on the basis of hydrophilicity. The methods described in several other papers, and software programs based thereon, can also be used to identify epitopic core sequences (see, for example, Jameson and Wolf, 1988; Wolf et al., 1988; U.S. Pat. No. 4,554,101). The amino acid sequence of these “epitopic core sequences” may then be readily incorporated into peptides, either through the application of peptide synthesis or recombinant technology.

Examples of epitopes that may be used to bind the ImmunoGlyph include peptides from, e.g., pancrease, L-asparaginase, hyaluronidase, chymotrypsin, trypsin, tPA, streptokinase, urokinase, pancreatin, collagenase, trypsinogen, chymotrypsinogen, plasminogen, streptokinase, adenyl cyclase, superoxide dismutase (SOD), and the like.

Examples of epitopes that may be used to bind the ImmunoGlyph include peptides from cytokines including, without limitation, interleukins, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Cytokines may be B/T-cell differentiation factors, B/T-cell growth factors, mitogenic cytokines, chemotactic cytokines, colony stimulating factors, angiogenesis factors, IFN-.alpha., IFN-.beta., IFN-.gamma., IL1, IL2, IL3, IL4, ILS, IL6, IL7, IL8, IL9, IL10, IL11, IL12, IL13, IL14, IL15, IL16, IL17, IL18, etc., leptin, my ostatin, macrophage stimulating protein, platelet-derived growth factor, TNF-alpha, TNF-beta, NGF, CD4OL, CD137L/4-1BBL, human lymphotoxin-.beta., G-CSF, M-CSF, GM-CSF, PDGF, IL-1.alpha., IL1-.beta., IP-10, PF4, GRO, 9E3, erythropoietin, endostatin, angiostatin, VEGF or any fragments or combinations thereof. Other cytokines include members of the transforming growth factor (TGF) supergene family include the beta transforming growth factors (for example TGF-.beta.1, TGF-.beta.2, TGF-.beta.3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

Examples of epitopes that may be used to bind the ImmunoGlyph include peptides from growth factors for delivery using the present invention include, without limitation, growth factors that can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

Examples of epitopes that may be used to bind the ImmunoGlyph include peptides from antigens such as viral antigens, bacterial antigens, fungal antigens or parasitic antigens.

Examples of epitopes that may be used to bind the ImmunoGlyph include peptides from viral antigens including, but are not limited to, e.g., retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins El and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS1, NS1, NS1-NS2A, 80% E, and other Japanese encephalitis viral antigen components; Abies viral antigens such as Abies glycoprotein, Abies nucleoprotein and other Abies viral antigen components. See Fundamental Virology, Second Edition, eds. Fields, B. N. and Knipe, D. M. (Raven Press, New York, 1991) for additional examples of viral antigens.

Viruses include picornavirus, coronavirus, togavirus, flavirvirus, rhabdovirus, paramyxovirus, orthomyxovirus, bunyavirus, arenavirus, reovirus, retrovirus, papilomavirus, parvovirus, herpesvirus, poxvirus, hepadnavirus, and spongiform virus. Other viral targets include influenza, herpes simplex virus 1 and 2, measles, dengue, smallpox, polio or HIV. Pathogens include trypanosomes, tapeworms, roundworms, helminthes, malaria. Tumor markers, such as fetal antigen or prostate specific antigen, may be targeted in this manner. Other examples include: HIV env proteins and hepatitis B surface antigen. Administration of a vector according to the present invention for vaccination purposes would require that the vector-associated antigens be sufficiently non-immunogenic to enable long term expression of the transgene, for which a strong immune response would be desired. In some cases, vaccination of an individual may only be required infrequently, such as yearly or biennially, and provide long term immunologic protection against the infectious agent. Specific examples of organisms, allergens and nucleic and amino sequences for use in vectors and ultimately as antigens with the present invention may be found in U.S. Pat. No. 6,541,011, relevant portions incorporated herein by reference, in particular, the tables that match organisms and specific sequences that may be used with the present invention.

Examples of epitopes that may be used to bind the ImmunoGlyph include peptides from bacterial antigens including, but are not limited to, e.g., bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diptheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumoly sin, pneumococcal capsular polysaccharides and other pneumococcal bacterial antigen components; haemophilus influenza bacterial antigens such as capsular polysaccharides and other haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens. Partial or whole pathogens may also be: haemophilus influenza; Plasmodium falciparum; neisseria meningitidis; streptococcus pneumoniae; neisseria gonorrhoeae; salmonella serotype typhi; shigella; vibrio cholerae; Dengue Fever; Encephalitides; Japanese Encephalitis; lyme disease; Yersinia pestis; west nile virus; yellow fever; tularemia; hepatitis (viral; bacterial); RSV (respiratory syncytial virus); HPIV 1 and HPIV 3; adenovirus; and small pox.

Examples of epitopes that may be used to bind the ImmunoGlyph include peptides from fungal antigens including, but are not limited to, e.g., candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Examples of epitopes that may be used to bind the ImmunoGlyph include peptides from protozoal and other parasitic antigens include, but are not limited to, e.g., plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

Examples of epitopes that may be used to bind the ImmunoGlyph include peptides from cell surface markers for dendritic cells include, but are not limited to, MHC class I, MHC Class II, B7-2, CD18, CD29, CD31, CD43, CD44, CD45, CD54, CD58, CD83, CD86, CMRF-44, CMRF-56, DCIR and/or ASPGR and the like; while in some cases also having the absence of CD2, CD3, CD4, CD8, CD14, CD15, CD16, CD 19, CD20, CD56, and/or CD57. Examples of cell surface markers for antigen presenting cells include, but are not limited to, MHC class I, MHC Class II, CD40, CD45, B7-1, B7-2, IFN-.gamma. receptor and IL-2 receptor, ICAM-1 and/or Fcgamma receptor. Examples of cell surface markers for T cells include, but are not limited to, CD3, CD4, CD8, CD 14, CD20, CD11b, CD16, CD45 and HLA-DR.

Examples of epitopes that may be used to bind the ImmunoGlyph include peptides from tumor antigens typically will be derived from the cell surface, cytoplasm, nucleus, organelles and the like of cells of tumor tissue. Examples of tumor targets for the antibody portion of the present invention include, without limitation, hematological cancers such as leukemias and lymphomas, neurological tumors such as astrocytomas or glioblastomas, melanoma, breast cancer, lung cancer, head and neck cancer, gastrointestinal tumors such as gastric or colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. Specific non-limiting examples of tumor antigens include: CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), Pmel 17(gp100), GnT-V intron V sequence (N-acetylglucoaminyltransferase V intron V sequence), Prostate Ca psm, PRAME (melanoma antigen), .beta.-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. In addition, the immunogenic molecule can be an autoantigen involved in the initiation and/or propagation of an autoimmune disease, the pathology of which is largely due to the activity of antibodies specific for a molecule expressed by the relevant target organ, tissue, or cells, e.g., SLE or MG.

Examples of epitopes that may be used to bind the ImmunoGlyph include peptides from antigens involved in autoimmune diseases, allergy, and graft rejection can be used in the compositions and methods of the invention. For example, an antigen involved in any one or more of the following autoimmune diseases or disorders can be used in the present invention: diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatibility antigens, and penicillin and other therapeutic drugs. Examples of antigens involved in graft rejection include antigenic components of the graft to be transplanted into the graft recipient such as heart, lung, liver, pancreas, kidney, and neural graft components. The antigen may be an altered peptide ligand useful in treating an autoimmune disease.

As used herein, the term “molecular proximity” refers to a molecular distance between 1-30 angstroms, namely, a distance in which two molecules can interact to trigger a reaction between the molecules. For example, a “molecular proximity” is the distance that extends between an enzyme and its substrate before, during, and after a reaction. In certain embodiments, molecular proximity is 1, 2, 3, 4, 5, 6, 7, 8, 9, 10, 11, 12, 13, 14, 15, 17, 20, 22, 25, 27, 30, 35, 40, 45, 50, 55, 60, 65, 70, 75, 80, 85, 90, 95, or 100 angstroms.

As used herein, the term “signal molecule” refers to a nucleic acid segment to which a detectable label is attached, e.g., a redox label, a fluorescent label, a metal, an enzyme, a chemiluminescent label, a magnetic or ferrous particle or a chromophore. There are many types of detectable labels, including fluorescent labels, which are easily handled, inexpensive and nontoxic.

As used herein, the terms “markers,” “detectable markers” and “detectable labels” are used interchangeably to refer to compounds and/or elements that can be detected due to their specific functional properties and/or chemical characteristics, the use of which allows the agent to which they are attached to be detected, and/or further quantified if desired, such as, e.g., an enzyme, radioisotope, electron dense particles, magnetic particles or chromophore. There are many types of detectable labels, including redox and fluorescent labels, which are easily handled, inexpensive and nontoxic.

Thus, when a signal molecule is adsorbed at the electrode surface it can be detected by one of several electrochemical methods. In the first type, a FET transistor is connected to the solution via an electrode to which the signal molecule adsorbs, in a form used in pH sensors. The adsorption of the signal molecule results in a change in the electrode-solution double layer capacitance which can be thought of as causing a threshold voltage change. Circuitry and auxiliary electrodes then bias the FET through the solution and the resulting current at a series of bias voltages can be used to quantify the adsorbed signal molecule. Quantification due to change in double layer capacitance can also be measured by using this setup to generate a CV or capacitance voltage curve utilizing small signal methods. Other methods utilize a potentiostat, these include but are not limited to Cyclic voltammetry, square wave voltammetry, differential pulse voltammetry, and AC voltammetry. In this embodiment, the signal molecule includes a redox label. The redox label has a certain electrochemical potential which can be thought of as a fermi level. The redox label will donate electrons to the electrode when its fermi level is negative of that of the electrode's fermi level. It will accept electrons from the electrode when the electrodes fermi level is negative of that of the redox label's fermi level. These techniques are based on utilizing various voltage forcing functions to bias the working electrode, which has the redox labeled signal molecules attached to it, with respect to the reference electrode. The various bias conditions result in a current, which flows between the working and counter electrodes. This generates a current-voltage numeric array which can be analyzed to quantify the amount of adsorbed species. The potentiostat can also be used to apply the open circuit potential method. In this method the quantity of adsorbed redox label alters the working-reference bias voltage, which results in zero current flow, a point which is called the “open circuit potential”.

This potential varies according the relative surface concentration of adsorbed redox label according to the Nernst equation and therefore can be used to quantify the amount of adsorbed species. In this embodiment, other redox couples may be added to the working electrolyte to initially poise the open circuit potential at a well determined point prior to introducing the unknown.

In one example, a “redox” cycle is measured by, e.g., a cyclic voltammetry, changes in pH, a potentiostat, an oxygen sensor, a resistance sensor, a capacitance sensor, or a sensor capable of detecting a change in the reduction or oxidation potential in a liquid. One such device is taught in U.S. Patent No. 9,176,087, which teaches an electrode that detects changes in a redox cycle using, e.g., standard hydrogen electrodes (SHE), normal hydrogen electrodes (NHE), reversible hydrogen electrodes (RHE), saturated calomel electrodes (SCE), copper-copper(II) sulfate electrodes (CSE), silver-silver chloride (Ag/AgCl) electrodes, or combinations thereof Another example of redox reactions includes reaction products that are detected with an electrochemistry cell, such as electrical signatures (cyclic voltammogram). In additional non-limiting examples, the reaction products may be those released from the cleaving of nucleotides from a nucleic acid or the addition of nucleotides to a nucleic acid strand such as that described in U.S. patent application Ser. No. 11/967,600, titled “Electronic Sensing for Nucleic Acid Sequencing,” relevant portions incorporated herein by reference. In another example, an oxide semiconductor is used as an electron accepting substance. More preferably, TiO₂, ZnO, SnO₂, Fe₂O₃, WO₃, Nb₂O₅, Ta₂O₃, In₂O₃, and strontium titanate indium-tin composite oxide (ITO), and/or fluorine-doped tin oxide (FTO) may be used. Non-limiting examples of electron accepting substances include, but are not limited to, elemental semiconductors such as silicon and germanium; oxide semiconductors of titanium, tin, zinc, iron, tungsten, zirconium, hafnium, strontium, indium, cerium, yttrium, lanthanum, vanadium, niobium or tantalum; perovskite semiconductors such as strontium titanate, calcium titanate, sodium titanate, barium titanate and potassium niobate; sulfide semiconductor of cadmium, zinc, lead, silver or stibium, bismuth; selenide semiconductors of cadmium or lead; a telluride semiconductor of cadmium; phosphide semiconductors of zinc, gallium, indium or cadmium; and/or compound semiconductors such as gallium arsenide, copper-indium-selenide and copper-indium-sulfide.

As used herein, the term “controller” or “processor” refers to an integrated circuit that is pre-programmed or is programmable to receive and process signals and to output these signals in a manner visible or understandable to a user, such as a print-out, display, sound, or other manner in which a user receives the results of the processing by the controller or processor. Typically, processors may transmit or receive data wirelessly via the one or more input/output interfaces. The one or more input/output interfaces can be any type of wired or wireless interface to other components, devices or systems either remote or locally located to the apparatus. The one or more input/output interfaces may be a display, a data storage, a printer, a communications interface, etc.

As used herein, a “Bioengineering Core Module” refers to an amino acid sequence of the beta barrel protein that serves as the scaffolding to create multi-epitope proteins.

As used herein, an “immunoGlyph” refers to multi-epitopes proteins formed by the insertion of the multiple (2, 3, 4, 5, 6, 7, 8, 9, 10, 11, 12, 13, 14, 15 or more) epitopes into the Bioengineering Core Module (BCM) that mimic regions of a pathogen or protein that are recognized by the immune system of a human observed as antibodies. A typical BCM is a beta barrel protein, which permits the insertion of peptides between the beta sheets of the protein, thus providing a stable protein that has multiple peptides that are on the surface of the protein and are thus accessible for binding (e.g., an antigenic peptide) and/or cleavage if the BCM is used in a reaction.

As used herein, an “ImmunoMimic” refers to multi-epitopes proteins formed by the insertion of the multiple (2, 3, 4, 5, 6, 7, 8, 9, 10, 11, 12, 13, 14, 15 or more) epitopes into the Bioengineering Core Module that serve as templates for the generation of antibodies that specifically bind to the epitope sequences.

ImmunoGlyph and ImmunoMimic production flow. Determination of the principle intended purpose of the protein. Capture antibodies (ImmunoGlyph), and/or Generating antibodies (ImmunoMimic).

For epitope selection and/or development: Recognition of sequences in peptide libraries by patient sera, peptide scans/arrays, and/or spot synthesis analysis.

For bioinformatics, use an epitope database and define uniqueness. The bioinformatics can also include structural information, e.g., X-ray crystallography of antigen complexed with antibody, computer modeling, phage display, and/or truncated resin-bound peptides/proteins.

Dyes or detectable markers include, but are not limited to, metal phthalocyanine such as copper phthalocyanine and titanyl phthalocyanine; chlorophyll or its derivatives; complexes of hemin, ruthenium osmium, iron and zinc (e.g., cis-dicyanate-bis (2,2′-bipyridyl-4, 4′-dicarboxylate)ruthenium (II)); organic dyes, such as metal-free phthalocyanine, 9-phenylxanthene dye, cyanine dye, metallocyanine dye, xanthene dye, triphenylmethane dye, acridine dye, oxazine dye, coumarin dye, merocyanine dye, rhodacyanine dye, polymethine dye, indigo dye and the like;

sensitizing dyes, such as, Cy3, Cy3.5, Cys, C_{y}5.5, C_{y}7, C_{y}7.5 and Cy9; AlexaFluor355, AlexaFluor405, AlexaFluor430, AlexaFluor488, AlexaFluor532, AlexaFluor546, AlexaFluor555, AlexaFluor568, AlexaFluor594, AlexaFluor633, AlexaFluor647, AlexaFluor660, AlexaFluor680, AlexaFluor700 and AlexaFluor750; and DY-610, DY-615, DY-630, DY-631, DY-633, DY-635, DY-636, EVOblue10, EVOblue30, DY-647, DY-650, DY-651, DYQ-660, and/or DYQ-661, for example.

For multi-epitope proteins, the design starts with amino acid sequence of the Bioengineering Core Module and the desired epitopes. For example, one or more of the following may be used to design the immunoglyph or immunomimic: match ends of the epitopes with potential insertion sites of the Bioengineering Core Module; addition of flexible linkers (Table 1); addition of balancing sequences; addition of purification motifs (FLAG, c-myc, 6×His, T7); addition of bioconjugation motifs (Lysines, cysteines); computer modeling to confirm that the addition of sequences for the epitopes do not have a detrimental influence on the predicted structure of the resulting protein, which should maintain the core structural elements of the bioengineering core module; and/or modification of sites that disrupt 3D structure.

Non-limiting examples of peptide linkers for use with the present invention include, for example, those listed in Table 1.

TABLE 1
Peptide Linkers

| Type | Sequence | Length | SEQ ID NO: |
|---|---|---|---|
| RigidD | APAPAPAPAP | 18.849 | 1 |
| Flexible | GGS | 6.114 | |
| Flexible | GGGGSGGGGS | 17.622 | 2 |
| Natural | EPKSCDKTHT | 21.628 | 3 |
| alpha helical | EAAAK | 6.297 | 4 |
| alpha helical | EAAAKEAAAK | 14.236 | 5 |
| alpha helical | EAAAKEAAAKEAAAK | 12.855 | 6 |
| alpha helical | AEAAAKEAAAKA | 16.319 | 7 |
| alpha helical | AEAAAKEAAAKEAAAKA | 21.387 | 8 |

Non-limiting examples of peptide tags for use with the present invention include, for example, those listed in Table 2.

TABLE 2
Peptide Tags

| Tag Type | Amino Acid Sequence | SEQ ID NO: | Isolation |
|---|---|---|---|
| Poly-His | HistiHHHHHHis9 | 9 | Ni2 + NTA, |
| | | | CO2 + CMAce |
| FlAsH t | agCOCCXXCCta10 | 10 | Bis-arsenical |
| | | | fluorescein dye |
| | | | FlAsHgs |
| HAT-a | KDHLIHVHLEEHAHAHN | 11 | CO2 + CMA |
| | Krell | | |
| Poly-Arg | | | Anionic resinsAH |
| Poly-Asp | re5oly-Aspresin | | Cationic resinsHN |
| Poly-Cys r4 aa (C) r | Poly-Cys r4 aa (C) r | | Thiopropyl |
| | | | Sepharosesc |
| Gluop1 aa (E)y1 | | | Cationic resinsar |
| Poly-Phe r11aa (E) r | Poly-Phe r11aa (E) r | | IECa |
| FLAG | TME)DYKDDDDKr | 12 | mAb M1, M2 M |
| c-myc | cMEQKLISEEDLes | 13 | mAb 9E10QK |
| T7E1 | MASMTGGQQMGsi | 14 | Anti-T7 9E10in |
| S-tagT7 | KETAAAKFERGHMDSar | 15 | S-proteinRG |
| Calmodulin- binding | yeKRRWKKNFIAVSAANR | 16 | CalmodulinVS |
| protein | FKKISSSGALny | | |
| Bio tag | liLGIFEAMKMEWRAA | 17 | Streptavidin/avidinIS |
| Strep-tag | diSAWRHPQFGGin | 18 | Streptavidin/a |
| Strep-tag II/ | WHPQFEKag19 | 19 | Strep-Tactin/a |
| Avi tag | acGLNDIFEAQKIEWHEid | 20 | Streptavidin/avidinIS |
| Nanotag | viDVEAWLGARdi | 21 | Streptavidin/avidinIS |

For cloning of the expression vector that produces the immunoglyph or immunomimic, one or more of the following may be used: back translation of final amino acid sequence into a DNA sequence, inclusion of restriction sites for cloning into expression vectors, exclusion of required restrictions sites within the coding region; codon optimization for the production host; and/or production of synthetic DNA.

Recombinant protein production can be accomplished in any of a number of expression systems, e.g., bacterial, yeast, mammalian, tobacco, carrots, insect cells, etc.

Certain advantages are obtained by using the present invention, including: presentation of the epitope within a whole protein, which improves its performance compared to the epitope as a peptide; presentation of the epitope on the surface of the protein to maintain interactions with antibodies in solution; the combination of multiple epitopes originating from multiple proteins into a single protein. Further, these advantages also include the ability to attach of one or more distinct and different epitopes into the same Protein bioengineering core module. This leads to a shortened development time due to the simplicity of combining epitope sequences into the sequence of the Protein bioengineering core module. Due to the flexibility of the system, it's possible to choose one or more epitopes of interest based on the end use: ImmunoGlyphs for applications that require the capture of antibodies as an indicator of health status and/or immunoMimics for applications that aim to generate antibodies with defined a performance. PAT production/Capture antibodies/Antibody pairs for capture and detection applications (i.e., Sandwich protocols).

The protein core is responsible for all biochemical aspects of production, including but not limited to: protein expression; protein solubility; protein purification; protein modifications; and/or protein stability. Epitopes that are responsible for immunological performance can include: binding antibodies; and/or generating antibodies.

The present invention provides a significant advantage over system of the prior art by the direct introduction of the sample will end the need for the user to manipulate or make any preparation of the sample, which diminishes the possibility for human error. The present invention is reaction-based/active vs. passive testing allows for extensive internal controls to ensure the performance of all components. The present invention allows for swarm-type detection in which all molecules of interest are examined, detected and bound in the same physical space, which permits a high level of multiplexing. The use of ImmunoGlyphs as the binding reagent will provide the highest selectivity and great sensitivity for detecting antibodies. The use of antibody pairs that bind the same MOI can be used to generate the Binding Reagent and part of the 1^{st} reaction (sandwich assay). Further, the present invention can be used capture proteinaceous components of a pathogen, which can rival the results from nucleic acid-based analyses. Each of the 4 steps in the ImmunoID process MOI binding; detection of MIO binding; generation of signal and signal detection are fast.

Further advantages of the present invention include a reaction-based analysis to generate a signal that is non-transient that allows the accumulation of signal and codification, which permits allocation of the measured signal to the identity of the MOI whose presence directed the reactions generating the signal that improves the robustness of the multiplexing. The present invention uses a solid-state measurements of the signal to eliminate end-user contributions to the interpretation of the results such that the user does not contribute to final results, which diminishes human error contribution. The solid-state method of measuring signals can include the use of one or more independent electrodes that allows the dedication of a subset to internal calibration of the system for signal intensity, signal production kinetics and signal position. Depending on the number of molecules of interest, the use of positive and negative controls, etc., the number of sensors on a chip or other signal detection interface can include 1, 2, 4, 6, 12, 48, 96, 486 or other number that will often mirror the types of plates commonly used in biological laboratories. However, the skilled artisan will immediately recognize that any number may be used, e.g., 1, 2, 3, 4, 5, 6, 7, 8, 9, 10, 15, 20, 25, 30, 35, 40, 45, 50, 60, 70, 75, 80, 90, 100, 200, 250, 300, 400, 500, 600, 800, 900, 1,000, 10,000, 100,000, 1,000,000, or more sensors per device. This is made possible using the novel immunoglyph system of the present invention in conjunction with standard semiconductor manufacturing processes.

The present invention has further advantages, include the production of signals, decoding/binding and measurement can occur simultaneously, which speeds up the process. A separate controller allows the digital distribution of the results. When used in the form of a closed system, a cartridge of the present invention can contain and secure any biologically hazardous samples. Further, the inclusion of inactivators, like sodium azide, is possible in the final reservoir of the waste fluids. It is also possible to use an absorbent, such as super absorbent polymer, that can transition biologically hazardous liquid material to a solid format. Used cartridges identified to contain scientifically relevant material could be used as a source of biological sample for downstream studies.

The present invention further leverages the natural phenomenon whereby two single-stranded DNA molecules that have complementary sequences will bind to each other with high specificity and sensitivity that is results in the non-transitory interactions observed as a stable binding. Two, non-complementary DNA sequences will only display transitory interactions that are short lived. The natural phenomenon can be extended to include single-stranded regions that are formed from non-naturally occurring biological mimics of DNA that include, but are not limited to, peptide nucleic acids, bridged nucleic acids and locked nucleic acids.

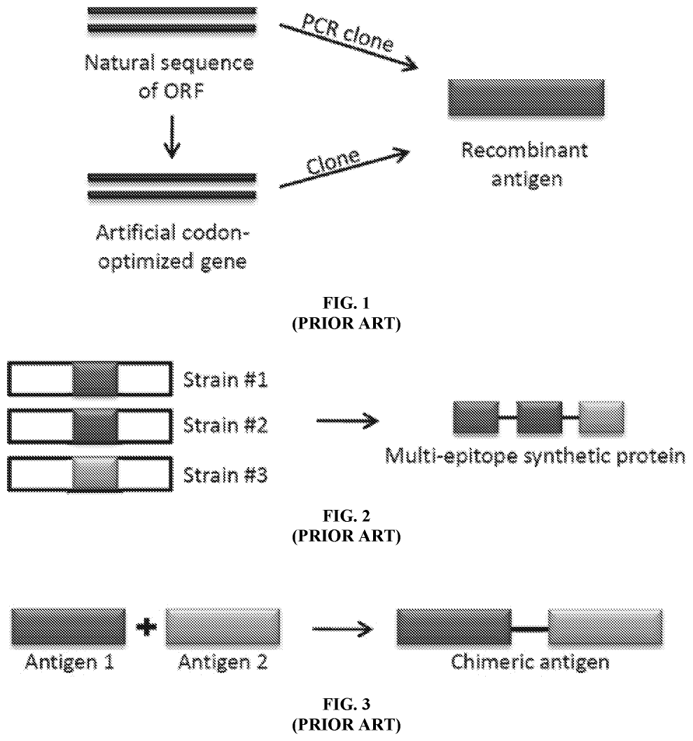

FIG. 1 shows one approach of the prior art, which is the transition to semi-purified proteins or recombinant production of proteins from a pathogen. FIG. 2 shows another approach of the prior art which is use of the most specific element of a pathogen for detection by antibodies are the short amino acid sequences of between 5-14 amino acids in the antigenic proteins of the pathogen, epitopes. FIG. 3 shows another approach of the prior art, which shows the inclusion of epitopes related to medically relevant in a subunit Chimeric/Fusion recombinant protein can present the epitope.

FIG. 4 shows the front-end chemistry and substrate of the present invention that includes includes an “immunoglyph” 2, which is tied to, e.g., micrometer sized magnetic beads or a planar non-magnetic substrate 1, but other substrates are possible. The magnetic bead approach allows the separation of solution chemistries from the beads by rinsing. With this approach, magnets can hold the beads while they are being rinsed. FIG. 4 shows what are termed mobile reaction centers, or MRC's. The immunoglyph 2 includes a functional group which is designed to capture a specific unique protein structure such as the variable region of an antibody or those which occur on the shell of a bacteria or surface of a virus. The MRC or substrate is depicted with a multi domain oligomer (MDO) which has five basic domains, domains 4-8. The first domain is a functional group 4, designed to attach to the surface of substrate 1 (e.g., a magnetic bead or planar substrate) with a surface chemistry 3. Examples of chemistry 3 include, but are not limited to streptavidin, noble metals, or silicon dioxide. For these examples, the corresponding functional group 4 would then be biotin, thiols, or silane groups, respectively. The attachment may or may not be covalent. For example, the surface of substrate 1 can be coated with streptavidin acting as the chemistry 3, which binds strongly to biotin, in this example functional group 4. This invention is not limited to the biotin/streptavidin attachment pair. A linking sequence or chemical molecule 5 attaches the functional group 4 to the second domain 6, which includes a base pair sequence that is designed to fold and be cleaved by a corresponding enzyme when the pathogen or antibody to be detected is present. The third domain of the MDO is a signal molecule 7, which contains a unique base pair sequence and a redox label, such as methylene blue as the signal 8. In this invention, the redox label options include but are not limited to methylene blue, ferrocene, and ruthenium bipyridine. When the target 9 (e.g., a pathogen, antigen, antibody, etc.) is detected this signal molecule 7 is cleaved from the MDO and is free to move about in the supporting solution or electrolyte used for back end electrochemical detection. In FIG. 4, the unknown 9, is the target molecule to be detected. The target molecule can be, e.g., an antibody, a bacteria, virus or other structured proteins, antigens, carbohydrates, lipids, small molecules, complex molecules, etc.

FIG. 5 shows the next step of the front-end chemistry, which is shown as a secondary antibody 10 conjugated to an enzyme 11 that binds the target 9. In the first example, an enzyme 11 is designed to methy late second domain 6 of the MDO targeted for cleavage. In another example, it is a restriction enzyme is used to cleave the target portion of the second domain 6 MDO after methylation. In FIG. 5, the unknown 9 has become bound to the immunoglyph 2. The immunoglyph 2 has a surface protein arrangement that is complimentary to and designed to spontaneously bind to a unique structure on the unknown 9.

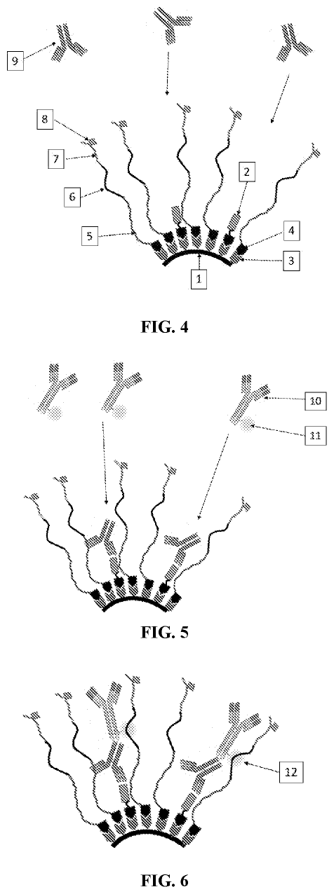

In FIG. 6 the next step in the chemical operation has been reached. The secondary antibody 10 has bound to the unknown 9. A sandwich consisting of the immunoglyph 2, unknown 9, and secondary antibody 10 has formed. Note that the secondary antibody 10 is now located so that the enzyme 11 is presented to the cleavable portion of the MDO complex 12.

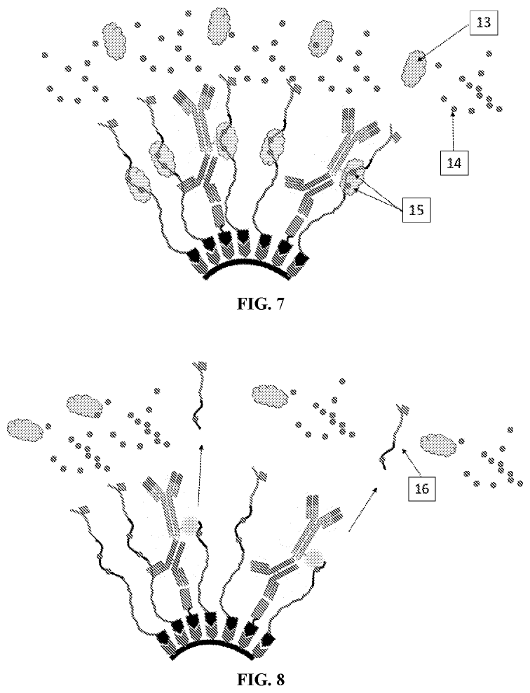

In FIG. 7, the last components of the front end chemistry 13-14 have been introduced. In either case, 14 is a source of methyl groups. In the first embodiment, 13 is a restriction enzyme whose role is to cleave the MDO complex 12 after the target sequence has been methylated. In this case, the enzyme 11, which is conjugated to the secondary antibody 10, is an enzyme 11 that methylates the target sequence 6. In a second embodiment, the roles of 11 and 13 are reversed: enzyme 11 is now the restriction enzyme, whose role is to cleave the MDO complex 12 and free the signal molecule 7-8 after the target sequence has been methylated. In this embodiment, enzyme 13 methylates the target sequence 6 to render it cleavable. As an example, enzymes 13 that catalyze methylation, might be, but are not limited to DAM (DNA adenine methyltransferase). An example of a methyl source is SAM (S-Adenosyl-methionine). The type of restriction enzyme chosen here is the sort, which can only cleave the target MDO base pair target sequence 6 after the appropriate bases have been methylated. In FIG. 7, the portion of the MDO 15 has been methylated, allowing cleavage to occur. Note that two enzymes, one for methylation, and the other for cleavage of the methylated target are required for cleavage. Cleavage cannot occur if methylation has not taken place. One of the enzymes is conjugated to the secondary antibody, this can be either the methylation or the restriction (cleavage) enzyme. The other enzyme is free in solution. These enzymes are added in different steps, separated by rinses. In order for both enzymes to be present together, one must be captured via the binding of the secondary to the primary antibody, otherwise it will be removed by the intermediate rinse. This scheme and sequence assures that only captured pathogens or antibody targets cleave to generate a signal. This is a key part of the invention. Other schemes can also be devised where cleavage requires two distinct chemical alterations, each carried out by different chemical constituents, where one is conjugated to an element which is captured in the presence of the target and the other free to move about in solution, can be devised and are covered as an alternate embodiment.

In FIG. 8, the signal molecules 16, have been cleaved, and are free to diffuse about in the supporting solution or electrolyte. These signal molecules 16 are the bridge between the front-end chemistry and back end detection, therefore they are a component of both the front end and back end chemistry. When a target 9 is present, the front-end chemistry frees the signal molecule 16 allowing them to be detected by the back end chemistry and supporting electrodes, electronics and software.

FIG. 9 shows the signal molecules 16, the capture molecules 18, the electrode 19, and the substrate 20. The back end chemistry includes the signal and capture molecules 16-18. These molecules are, e.g., short DNA strands. The substrate 20 contains many electrodes 19, each if which are functionalized with unique capture molecules 18. DNA strands will hybridize only when the capture 18 and signal strands 17 are exactly or mostly complimentary (depending on the conditions selected for binding). Because of this the front-end chemistry is designed to look for several pathogens in parallel.

When a bead substrate approach is utilized (e.g., magnetic beads), different sets of beads are each functionalized with a unique MDO-immunoglyph combination. The MDO then has a unique signal molecule base pair sequence corresponding only to the pathogen that its partner immunoglyph is designed to capture. Combining different bead sets allows several unknowns to be screened for in parallel. When the planar approach is used, the front-end chemistry plane contains several domains. Each domain is functionalized with a unique immunoglyph MDO combination depending on the desired set of unknowns to be screened for in parallel.

The Mobile Reaction Centers (MRC) can be coated with a capture reagent for Molecule of Interest (MOI)-A or MOI-B, each with a corresponding chemical compound with a section unique to and correlating to either A or B that will serve as a measurable signal. The reaction centers can be mobile or non-mobile. Reaction centers can be composed of a variety of substances including but not limited to, paramagnetic particles, glass and plastic. The capture agent is chosen based on its ability to bind the molecule of interest. In the case of detecting antibodies that represent an infection by a pathogen, the binding agent can be a bioengineered multi-epitope protein. In the case where the MOI is a pathogen, the binding agent can be an antibody that specifically binds the pathogen. The chemical compound, in one configuration, is made from a string of nucleic acids (A, C, G, T). The sequence of the nucleic acids is uniquely associated with the identity of the MOI. Attachment to the surface of the paramagnetic particle can be through a variety of chemical linkages or even biotin-streptavidin interactions.

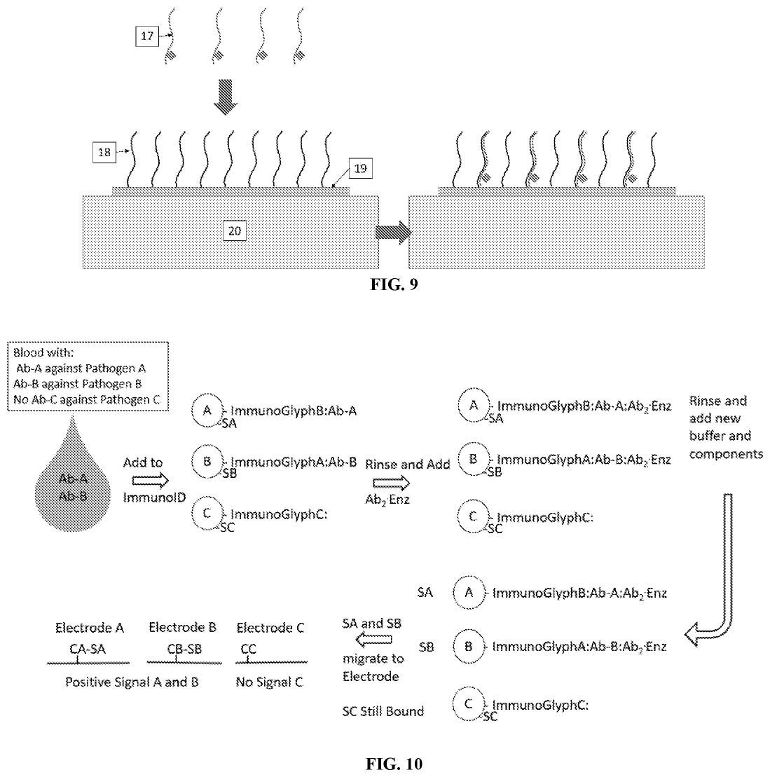

FIG. 10 is a representative flowchart of one embodiment of the present invention for the detection of antibodies that recognize and bind to components of pathogen A and B, but not pathogen

C in a sample of patient blood as an indicator of whether the patient is/was infected by pathogen A, B or C. A biological sample of the patient's blood is introduced into an ImmunoID diagnostic test system. The system contains three classes of mobile reaction centers built on paramagnetic particles consisting of a binding reagent (A, B or C) to capture antibodies and a chemical compound (A′, B, or C′) with a strict correlation to the binding reagent such that A & A′ are associated, B with B′ and C with C′. The binding reagents can capture the antibody against their respective pathogen (Pathogen A, Antibody (Ab)-A, Binding Reagent A, Chemical compound A′; etc.). After mixing the patient sample with the mobile reaction centers, if antibodies against the pathogen are present, they will bind the binding reagent (the hypothetical sample of the figure contains Ab-A and Ab-B, but not Ab-C). If a person had not been infected by a pathogen, no antibodies against that pathogen will be present and none will bind the binding reagent representing that pathogen (Ab-C). Next, electromagnets can be used for non-contact movement of the mobile reaction centers to a new region/well of the system that allows interaction with a unique biochemical reagent consisting of a secondary antibody, which binds human antibodies, and is conjugated to an enzyme that can act upon the chemical compounds on the surface of the mobile reaction centers. After a period of time (˜5 min), fluidic controls can change the solution around the mobile reaction centers to remove the secondary antibodies conjugated with the enzyme to include accessory proteins/enzymes/chemical compounds to the enzyme conjugated to the secondary antibody. The presence of these accessory elements along with the bound secondary antibody, which is due to the presence of antibodies against a particular pathogen, will allow the modification of the associated chemical compound such that a portion of the compound (designated as A″ and B″) are released and serve as signals. No C″ is released due to the absence of antibodies against pathogen C that prevents the association of the secondary antibody. The released elements of the chemical compound can translocate, through diffusion, mixing motion, electrical forces, to a physically separate region/compartment/well that has an array of electrodes where each electrode was functionalized with a chemical compound complementary to the released elements (designated as A°, B° and C°). If signal elements are released, they can be bound by the complementary compound, which can be detected and measured by a change in the microenvironment of the electrode that alters its physical properties such as current or voltage.

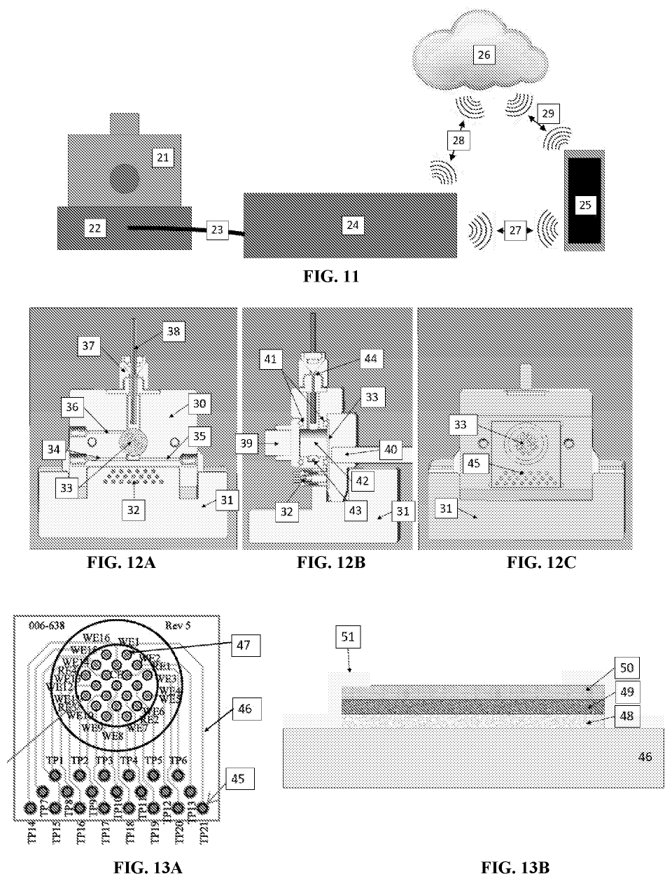

In FIG. 11, the role of the cartridge 21 is to facilitate the various chemical steps discussed previously. The cartridge, detailed in FIG. 3, that may include a number of wells 42, 43, or enclosed volumes which contain the chemistry. The device can include ports 34-36, which facilitate the transfer of chemical reagents 34, supporting electrolytes 35, and rinse water 36 into and out of the wells. In the example three ports are shown, but more can be utilized. The liquids are transferred or driven by a pump, e.g., a syringe or other type of pump The syringes may be external to the cartridge and connected via a coupling at the outside end of chemical reagents 34, supporting electrolytes 35, and rinse water 36. In another embodiment, the syringe or syringe pump may be incorporated directly into the body of the cartridge. The syringes can be driven by an electronically controlled actuator, for example a stepper motor and lead screw. The cartridge body is constructed of plastic and may be fabricated by machining, injection molding, 3D printing, or other suitable methods. One novel aspect of this approach is that it does not require valves due to the small port diameter, hydrophobicity of the plastic and closed piston design of the syringe pumping system. In order for fluids to be displaced there must be a pathway for the gas contained in the fluid paths to migrate through. Because the system is closed, it ends in a sealed piston, there is no pathway for gas or fluid to escape. Also, because the diameter of the pathway is small and the surface of the pathway is hydrophobic, force must be applied to form a new liquid surface. This force must overcome the surface tension of the liquid and lack of wettability of the plastic body which increases the surface energy. The syringe or piston pumping force is great enough to overcome this, but gravity is not. This approach, which avoids the need for valves, reduces the manufacturing cost of the cartridge.

The cartridge includes a circuit array. This provides an array of electrodes 33 used in the electrochemical measurement and can generate an electrical signal into and out of the well 45. Electrodes 33 and 45 are tied together by a electrically conductive interconnect shown as 46 in FIG. 13A. The interconnect is covered by a hydrophobic patterned dielectric such as polyimide 51. Other patternable dielectric layers such as, but not limited, to Paralyene, or liquid photo-imageable hard mask can also be utilized. Openings in the dielectric are formed to above the electrode surface 33 and contacts 45. The cartridge contains and forms a well around the electrical connecter array 32. There are three types of electrodes in the array, working electrodes, which hold the capture molecules, reference, and counter electrodes. These are used in a typical 3 electrode electrochemical cell. During electrochemical measurements, the well is filled with a supporting electrolyte, such as 1X Phosphate Buffered Saline (PBS). The cartridge 21 can include one or more o-rings 42, which form a seal between the plastic cartridge body and the electrode array. An injection port 37 provides a sample injection 38, for example, by a capillary. The cartridge is designed as a sealed system and the injection port allows samples to be introduced but prevents contained liquids from escaping by gravity. This aids in preventing operators from being exposed to harmful pathogens or biohazards contained in the patient sample. The injection port includes an elastomer 44 with a small hole in it. The elasticity allows a larger diameter sample injection instrument such as a capillary, needle, or pipette to pass through the smaller hole and also closes the hole upon removal of the sample injector. The combination of the hydrophobicity of the elastomer and small hole size prevent liquid escape by gravitational forces, again utilizing the principle of an energy barrier provided by surface tension and hydrophobicity. In one embodiment, the cartridge is supplied with all necessary liquids contained within the syringe pumps. Any liquids which are removed in a given step are contained within syringe pump bores avoiding contact of biohazard liquids with the operator. Initially, before use, these liquids may be contained directly within the syringe bore, on placed inside a capsule or plastic bag which is contained in the bore. The role of the capsule would be to provide a sealed environment for the supporting electrolytes and reagents so that shelf life is increased. The encapsulation process can be any of those well-known in the pharmaceutical industry. In the case where electrolyte capsulation is used, a sharp edge would be included in the syringe bore so that compressing the piston would rupture and release the contents of the capsule. Reagents which are more stable in dried form would be freeze dried on the appropriate well surface, for example 42. In FIG. 11, the cartridge 21 plugs into a connector 22, which provides electrical interconnect between the controller and cartridge. The connector includes contacts 32 which make electrical contact with the array. Bores 39-40 are included which provide close access for fixed or electromagnets used for separation of rinse eluent and magnetic bead (MRC or mobile reaction center) front end approach. When the planar front-end substrate is used magnets are not required. The well volume is reduced in order to facilitate mass transport of the signal molecules to the capture molecule functionalized electrodes. In a particularly elegant embodiment, the planar front end substrate is placed on the face opposite of the array and the dimension between them minimized forming a thin layer cell. This increases the sensitivity of the signal response and therefore reduces measurement time by increasing the signal concentration due to volume reduction and mass transport due to increased concentration and reduced diffusion length between the source MDO and electrode planes. FIGS. 12A to 12C show a cartridge embodiment with a vertical orientation. This is beneficial for avoiding false positive signals that occur due to uncaptured front end substrate particles. In a vertical orientation these particles will tend to fall to the well bottom which is benign in a vertical orientation, but results in false positive signals in a horizontal configuration where the array electrodes are on the bottom of the cell. Other orientations are possible but less desirable, for example, a horizontal configuration with the array on top would also avoid the false positives but would be problematic due to air bubbles collecting on the electrode surface.

The array is shown in FIG. 13, comprise one thin film conductor interconnect path starting at a pad 45, that connected via conductive line or path 46 to a electrode 47. The line 46 denotes the path 46, which is terminated by a pad on each end. Pad 45 is a connection point to the connector tied to the electronic controller through wires 23 in FIG. 11. The electrode 47 is the electrode end, which can serve as a working, reference, and/or counter-electrode, or can include all three. The working electrode 19 (see FIG. 9) is where the capture molecules 18 attach. For example, thin film conductors are covered with a hydrophobic patterned dielectric 51, for example polyimide. The dielectric has openings patterned over the pads which terminate the conductor ends. In order to functionalize the working electrode with capture molecules, a small drop of solution containing the capture molecules and other necessary reagents is deposited on the working electrode. Size reduction requires a high density of parallel working electrodes, each functionalized with a different capture molecule type. The drops containing the capture molecules must be kept separate during functionalization, which requires the dielectric surface to be hydrophobic to prevent the drops from spreading. The metal stack starts with a glue layer, 48, which has to be reactive in order to adhere to the substrate, for example titanium, chrome, or tantalum. High reactivity is important because any glue layer metal which is exposed to the electrolyte for example by diffusing through the stack, forms a highly stable oxide. A highly stable oxide will not be electrochemically active in the potential ranged used to detect the signal molecule redox label. The remaining metals are noble, that is, they do not react with oxygen and their redox potentials are positive of the potential range used for signal molecule redox label detection. Examples are gold, platinum, palladium, iridium, and rhodium. When titanium is used as a glue layer, there is usually an intermediate layer 49 used to facilitate adhesion in harsh environments, for example palladium or platinum. Typical currents are small, in the nanoamp range, therefore the metal path resistance can be as high as several megaohms without significantly affecting the measurement. This allows noble metal stack thicknesses as low as 2000 angstroms which reduces cost. Ceramic substrates such as Alumina can be used 46, but other materials such as FR4 and other organic substrates are possible and provide a lower cost option.

The actual measurement sequence is automated and controlled by a control module 24 in FIG. 11. The control module consists of a microcontroller, WIFI, Bluetooth, and LORA radio links, a battery, power management, a potentiostat, and various multiplexors required to access the various electrodes for individual measurement. FPGA's may be included to facilitate high speed control. A potentiostat is the analog interface lies between the microcontroller and electrochemical cell. The controller applies one of several possible electrochemical methods to the potentiostat for redox label detection, for example but not limited to Square Wave Voltammetry, AC voltammetry, Cyclic Voltammetry, Differential Pulse Voltammetry, open circuit potential, or capacitance techniques. Control is distributed between the microcontroller located in the control module 24 and the host 25. The host may be a personal computer or smartphone. Wireless communication links between the host, the controller and cloud are included. The host has several software layers while the microcontroller has one layer of embedded code. The host initiates each step of the measurement process, which is carried out by the microcontroller. After the controller completes each measurement, it sends raw data to the host for post processing. Prior to measurement processing, the data may be noise filtered by software using methods including, but not limited, to median filtering and spline fitting. After measurement post processing the host presents the results to the user and uploads the data to a database repository in the cloud. The data packet includes physical location information which can allow for tracking of infection patterns by data base mining. The controller also includes, e.g., a LoRa® path to the cloud 28 for applications where WIFI connectivity to the clouds is not available.

Table 1 is a list of potential types of antibodies that can be detected in the blood of a patient together how they are informative of the patient's health.

| | |
|---|---|
| Antibody Classes | Type of Response |
| IgG | Primary Response |
| IgG subclasses | More detailed information on the immune |
| | response to an infection |
| IgM | First response |
| IgE | Allergic response |
| IgA | Mucus membrane |

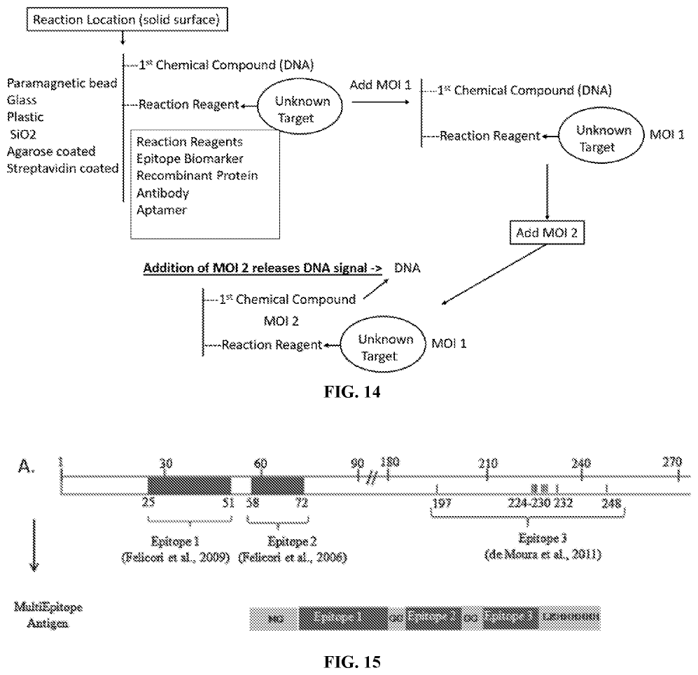

FIG. 14 is an illustration of the combination of a reaction reagent with its corresponding 1^{st} chemical compound on the surface of a solid substance. Multiple substances can contribute the surface including, but not limited to, paramagnetic particles, glass, plastic, silicone, and agarose. The reaction reagent can bind the MOI and can be, but are not limited to a multi-epitope biomarker, recombinant protein, antibody and aptamer. Following the potential introduction of the MOI, a series of reactions (labeled 1, 2 . . . ) results in the release of the 1^{st} chemical from the solid surface that is freely mobile and serves as a signal, which can interact with a 2^{nd} chemical compound. Binding of the signal to the 2^{nd} chemical compound results in a measurement. Two different series of reactions are shown that can result in the release of the signal as an indication for the presence and capture of the MOI. In one series, the first reaction is the activity of a DNA methyltransferase that can modify with a methyl group a specific sequence of base pairs in double stranded DNA, which can serve as the 1^{st} chemical compound. The second reaction is the activity of a restriction enzyme that recognizes the same sequence as the DNA methyltransferase and can create a break in the DNA, but only if the sequence is methylated. In another series, the first reaction places ½ of a heterodimeric restriction enzyme in proximity of the 1^{st} chemical compound and the second reaction delivers the other half of the heterodimeric pair, which results in the breakage of the DNA and release of signal.

FIG. 15 shows that the natural sequence of a protein can be replicated for recombinant protein production or the sequence can be codon optimized for the production host for recombinant protein production. An epitope found in a protein can have slight differences between strains that can be captured and incorporated into a single, multi-epitope protein formed by the linear arrangement of the epitopes without regards the form of the final protein. Panel C shows that epitopes from two different proteins can be combined into a chimeric protein of unknown biophysical and biochemical characteristics.

Numerous proteins in the Protein Data Bank (PDB) describe proteins that contain multiple beta-sheet segments, both parallel and antiparallel, which fold into a barrel shape. The structure of these barrel structures can be described by the geometric parameters of the number of strands and the shear number, which is a measurement of the stagger observed between the strands around the barrel. There are two general classifications, closed/complete barrels and open/distorted barrels. A closed barrel can be identified by a complete ring of hydrogen bonds in the secondary structure. To serve as a bioengineering core modular in the design of ImmunoGlyphs, the 13-barrel will be modified by the insertion of epitope sequences into the regions between the 13-sheets that comprise the strands. Table 3 lists the different sizes of known barrel proteins with their range in shear numbers along with an example. An exemplary example of a β-barrel family of proteins to serve as the bio-engineering core modular are the fluorescent proteins that have 11 strands and a shear number of 14. Non-limiting examples of beta barrel proteins are listed in Table and the sequences of the same are incorporated herein by reference.

TABLE 3

| # of Strands | Shear # | # of Proteins | Example |
|---|---|---|---|
| 4 | 8 | 6 | Neurotrophic Factor |
| 5 | 8 to 12 | 87 | Ribosomal protein L14 |
| 6 | 6 to 12 | 795 | Thrombin |
| 7 | 10 | 14 | Pyruvate kinase |
| 8 | 6 to 12 | 334 | Enolase |
| 11 | 14 | 132 | GFP |
| 14 | 14 | 1 | alpha-hemolysin |
| 16 | 20 | 3 | Porin |
| 18 | 22 | 16 | Maltoporin |
| 20 | 20 | 4 | GTP cyclohydrolase 1 |

Table 4: Association of the Electrode position with the sequence of the Region 4 (signal portion) that can be associated to the binding reagent. As the 1st chemical compounds are oligonucleotides, the portion that is released and serves as a signal can have a sequence that is uniquely registered to the identity of the MOI captured by the Binding Reagent that is associated with it on the mobile or non-mobile Reaction Center. In addition, the sequence uniquely registers the signal to a specific electrode functionalized with an oligonucleotide that has its complementary sequence. As consequence, the measurements made at the electrode are directly relatable to the MOI based on the shared association with the signal sequence.

TABLE 4
Association of the Electrode position with the sequence of the Region
4 (signal portion) that can be associated to the binding reagent.

| | | Binding Reagent | | | |
|---|---|---|---|---|---|
| | Region 4 | | Auto- | | |
| Electrode | (in FIG. 10) | Infection | Antibody | Pathogen | TBD |
| 1 | Sequence 1 | Pathogen A | Disease A | A | MOI 1 |
| 2 | Sequence 2 | Pathogen B | Disease B | B | MOI 2 |
| 3 | Sequence 3 | Pathogen C | Disease C | C | MOI 3 |
| N | Sequence N | Pathogen X | Disease X | X | MOI-X |

Sequences 1-N can be mixed with any combination of binding reagents to generate a cartridge based on the MOIs to be tested. In practice, a look-up table is generated to track the relationship between the sequence of the signal molecule, the MOI and the electrode position that will capture and measure the signal molecule. The look-up-table that is available through an associated barcode or some other parsing mechanism (on system, cloud-based, other). An advantage to the system is that the array of electrodes can be functionalized in the same manner for use in any cartridge independent of the final list of MOI, which simplifies manufacturing. The MOI tested are determined by the associations generated by mixing the Binding Reagent with 1st chemical compound.

Detecting patient antibodies (infectious disease, autoantibodies), outlined in FIG. 2 The use of an ImmunoGlyph as the binding reagent will give very highest specificity and sensitivity in comparison to other immunological reagents for detecting antibodies made in response to an infection or rogue self-protein (autoantibodies)

Detecting microorganisms (surface, water-borne), similar to detecting patient antibodies. The ImmunoGlyphs are replaced as the binding reagent by an antibody that can capture the microorganism of interest. Also, the 1st reaction antibody-enzyme conjugate has the antibody against human IgG or IgM replaced by secondary antibody/antibodies that also recognizes the microorganism. This technique is commonly referred to as a “sandwich assay”. (Note: an antibody can be a monoclonal or a polyclonal).

Detecting individual proteins is the same as the microorganism, only with a pair of antibodies that both recognize the protein of interest.

Internal Controls to report on the performance of an individual test cartridge.

Negative controls (no reaction reagent with a 1st chemical compound; direct inclusion of signal in the reaction well to measure carryover between compartments).

Positive controls (detection of antibodies in the sample; detection of the antibody-enzyme conjugate; detection of the enzymatic reaction; direct inclusion of signal in array well).

Calibration controls for software analysis of the results that include, but are not limited to detection of peak position, peak intensity, measurement offset. Multiple multi-domain oligos (1st chemical compound) can be combined in different molar ratios at a single Reaction Center to generate calibration curves for signals at high, medium and low levels. The oligos would be preconditioned to release signal molecules in the final, array chamber exclusive of upstream events. The signal from the highest concentration can also be used to define the voltage position in the software of the peak current levels used to automatically detect positive signals.

Determination of the sufficiency of the sample size introduced for testing Patient blood samples to provide results of high confident. A reaction center (mobile or non-mobile) can be included with a binding reagent to detect the quantity of antibodies that enter the system that could include Protein A, G or L, specific antibodies against human antibody classes (IgG, IgM, IgA, IgE) or subclasses. The intensity of the signal compared to the calibration curve can be used to define a threshold for the minimum quantity of antibodies necessary to perform the test. If the readout is below the threshold, then the test will indicate that an insufficient volume of patient sample was added to the cassette.

Organization of the cartridge. Methods to transition MOI into aqueous solutions. Introduction of samples. Measurement technique

Additional information on ImmunoGlyphs and ImmunoMimics: The correlation between an antibody and the antigen it binds has a tremendous usefulness for numerous applications. One aspect for protein antigens, the smallest segment comprising its amino acid sequence that is bound by the antibody is known as an epitope, which consists of 4-14 amino acids. For immunological applications, the combination of multiple epitopes into a singular recombinant protein can provide reagents of high utility. To date, no uniform methodology has been developed for generating recombinant proteins containing multiple epitopes. Here, a method is provided to generate proteins containing a large number of epitopes based on the incorporation of the epitope sequences into a protein core derived from a beta-barrel protein. The core sequence contributes biochemical characteristics to the final multi-epitope protein for ease in design and production while the epitopes incorporated in the core sequence contribute the immunological characteristics.

Uses of the ImmunoGlyphs and ImmunoMimics: (1) Represent/mimic pathogens to bind pathogen-specific antibodies: A. Diagnostic, b. Prognostic, and/or c. Purification of antibodies. (2) Represent/mimic pathogens to stimulate the production of antibodies: A. Vaccines that target the production of antibodies to specific regions of a pathogen or pathogen produced toxins. i. Inclusion of all subclass variations. (example of three sites in rabies that can neutralize the virus, but a single amino acid difference makes an antibody ineffective, which is observed between the three major variants in global circulation. ii. Design the inclusion of immunological boosters. B. For the production of Passive antibody therapies (PAT) by using the protein as the antigen to induce the immunological response of the antibody donor. i. Improve the specific activity of PAT by only including the epitope targets/sites that provide therapeutic benefits. Unnecessary sequences are removed. ii. Improve production levels by eliminating immunological responses to non-therapeutic components. iii. Improved safety by eliminating the need for producing biologically active pathogens. C. Performance-defined antibodies that target a unique site in a protein of interest for generating specific antibodies. A method to focus the antibody binding site in a target. D. Generation of antibodies against surface protein biomarkers of cancer cells that can be used to target toxins and/or visualization markers.

Isolate and amplify regions of interest in proteins of interest for the production of antibodies that target that desired region: including, immunological targeting of cancer therapeutics, immunological targeting of cancer visualization markers, differentiate closely related proteins, or user designated regions of interest. It is also possible to represent epitopes within endogenous proteins that are recognized by autoantibodies, e.g., Diabetes, multiple sclerosis (MS), and/or rheumatoid arthritis.

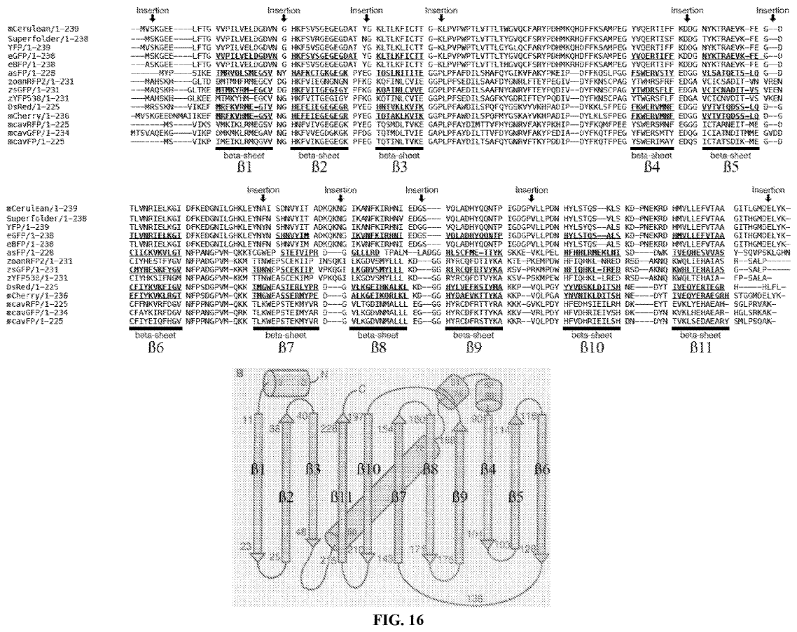

Detailed embodiments of the use of the patent to generate immunological reagents. The amino acid sequence of a Barrel or Cylinder Proteins (FIGS. 16 to 18B) shows that beta-barrel or cylinder proteins can serve as a bioengineering core module that is used in conjunction with the manipulation of its coding DNA sequencing to include new stretches of amino acids that represent the sequences of epitopes that are specific to a pathogen or protein that will bind to antibodies generated against the pathogen or protein. Beta barrel proteins are grouped by the number of staves (Beta-sheet segments) that form the barrel. Some beta barrel proteins form pores in lipid bilayer membranes and should be avoided as the core sequence. There are 1172 sequences of beta-barrel proteins in the Protein Data Base (PDB). A subset of beta barrel proteins are naturally unexposed to humans and do not display an inherent reactivity to normal patient sera. The present invention can be adapted to use any of the beta barrel proteins.

Epitopes identified for a specific virus (or pathogen) can provide manipulation of specific strands of DNA sequences which will create a virus protein analogy that will specifically attach to an antibody. Since the epitopes (FIG. #20) that are created are limited only to the specific virus, a high degree of specificity is created and only allows for binding of specific antibodies.

Example 1. The detection of HIV infection based the incorporation of multiple epitopes into Thermal Green Protein that serves as the bioengineering core module. The following epitopes were identified as unique to HIV-1 and recognized by antibodies in serum from HIV-1 infected patients:

1.
SEQ ID NO: 22
PTKAKRRVVQREKP
(gp120)
2.
SEQ ID NO: 23
GCSGRLICTTNVPW
(gp160)
3.
SEQ ID NO: 24
LLSSWGCKG [symbol] VCYTSVQWNET
(gp41)
4.
SEQ ID NO: 25
LLSLWGCRG [symbol] VCYTSVQWNET
(gp41)
5.
SEQ ID NO: 26
RILAVERYLKDQ
(gp41)
6.
SEQ ID NO: 27
RLLGIWGCSGKLICTT
(envelope glycoprotein)
7.
SEQ ID NO: 28
RALETLLNQQRLLNSWGCKGRLVCYTSV
(gp41)
8.
SEQ ID NO: 29
NTRKSIRIGPGQTFIA
(envelope glycoprotein)
9.
SEQ ID NO: 30
RKSVHIGPGQAFYA
(pg120)

Of interest is that epitopes 3 & 4 represent observed polymorphisms that only differ by two amino acids. In addition, the spacer sequences GGSG SEQ ID NO: 31 & GGGASG SEQ ID NO: 32 were included to provide a section of flexibility. Lastly, two proteins are included. The 6-HIS tag that allows purification through immobilized metal affinity chromatography and three lysines to permit the attachment of bioconjugates through NHS-chemistry.

HIV1-ImmunoGlyph 1, HIV Epitopes are bolded, spacers are underlined, tag is italicized, betabarrel is bold and underlined.

SEQ ID NO: 33
MGAPTKAKRRVVQREKPHASVIKPEMKIKLRMEGAVNGHKFVIEGEGIGK
PYEGTQTLDLTVEEGCSGRLICTTNVPWGGSGAPLPFSYDILTPAFQYGN
RAFTKYPEDIPDYFKQAFPEGYSWERSMTYEDLLSSWGCKGRLVCYTSVQ
WNETQGICIATSDITMEGGSGLLSLWGCRGKAVCYTSVQWNETDCFFYEI
RFDGTNFRILAVERYLKDQKKTLKWEPSTEKMYVEDRLLGIWGCSGKLIC
TTGGSGVLKGDVEMALLLEGGRALETLLNQQRLLNSWGCKGRLVCYTSVG
HYRCDFKTTYKAKKDNTRKSIRIGPGQTFIAAHEVDHRIEILSHDKRLNS
WGCKGRLVCYTSVDYNKVRLYEHAEARYSGGGSGGGASGRKSVHIGPGQA
FYAHHHHHHKKK

The sequence was analyzed to provide a predictive three dimensional model of the resulting protein at zhanglab.ccmb.med.umich.edu/I-TASSER/output/5357812/8zeuez/. From the model, it was clear that the inclusion of GGGSGGGASG was not helpful due to its formation into an additional 13-sheet that altered the core 13-barrel. After truncating to GGSG, the resulting 3D computer model showed a structure similar to the core TGP segment with the epitopes free and at the surface.

HIV-ImmunoGlyph 2, mature protein

SEQ ID NO: 34
GAPTKAKRRVVQREKPHASVIKPEMKIKLRMEGAVNGHKFVIEGEGIGKP
YEGTQTLDLTVEEGCSGRLICTTNVPWGGSGAPLPFSYDILTPAFQYGNR
AFTKYPEDIPDYFKQAFPEGYSWERSMTYEDLLSSWGCKGRLVCYTSVQW
NETQGICIATSDITMEGGSGLLSLWGCRGKAVCYTSVQWNETDCFFYEIR
FDGTNFRILAVERYLKDQKKTLKWEPSTEKMYVEDRLLGIWGCSGKLICT
TGGSGVLKGDVEMALLLEGGRALETLLNQQRLLNSWGCKGRLVCYTSVGH
YRCDFKTTYKAKKDNTRKSIRIGPGQTFIAAHEVDHRIEILSHDKRLNSW
GCKGRLVCYTSVDYNKVRLYEHAEARYSGGSGARKSVHIGPGQAFYAHHH
HHHKKK

From the amino acid sequence, it was possible to determine the expected molecular weight (45903.44 daltons) and its theoretical pI (9.04). Next, the sequence was reverse transcribed to give a DNA sequence that was codon optimized for the expression host (E. coli) and devoid of restriction sites for downstream molecular biology techniques.

HIV-ImmunoGlyph Nucleic Acid Sequence

SEQ ID NO: 35
ATGGGAGCCCCCACGAAGGCAAAGCGTCGTGTTGTCCAACGTGAGAAGCC
ACATGCGAGCGTTATCAAGCCTGAAATGAAAATTAAACTGCGCATGGAGG
GGGCAGTAAATGGGCACAAGTTTGTCATTGAGGGGGAAGGGATTGGCAAA
CCTTACGAAGGGACCCAGACTCTGGACTTGACGGTGGAGGAGGGGTGCAG
TGGCCGCCTTATCTGTACCACGAATGTCCCATGGGGTGGCTCTGGCGCCC
CCCTTCCTTTCTCCTACGATATTCTTACGCCGGCGTTTCAGTACGGGAAC
CGTGCTTTCACCAAGTACCCAGAGGATATTCCAGACTACTTTAAGCAGGC
TTTCCCCGAGGGGTACTCATGGGAGCGTTCAATGACGTACGAGGATCTGC
TTAGTAGCTGGGGTTGCAAAGGGCGTCTTGTTTGCTACACCTCCGTGCAA
TGGAACGAGACGCAAGGGATTTGTATTGCTACCAGTGACATCACAATGGA
AGGGGGGTCCGGGTTGTTATCTTTGTGGGGGTGCCGTGGCAAGGCAGTAT
GCTATACGAGTGTACAGTGGAATGAAACAGATTGCTTTTTTTATGAAATC
CGCTTTGATGGTACAAATTTCCGTATCTTGGCGGTTGAACGCTATTTGAA
GGATCAGAAGAAGACTTTGAAATGGGAGCCATCCACTGAAAAAATGTACG
TAGAGGATCGCTTGTTAGGAATTTGGGGCTGTAGTGGCAAATTGATTTGC
ACTACTGGCGGAAGCGGTGTCCTTAAGGGTGACGTAGAAATGGCCTTACT
TTTAGAAGGCGGCCGCGCTTTAGAAACCTTGCTTAATCAGCAGCGCCTTC
TGAATTCATGGGGGTGTAAAGGACGCTTGGTGTGTTATACGTCCGTGGGC
CATTATCGCTGCGATTTTAAGACTACGTACAAGGCAAAAAAGGATAATAC
CCGCAAAAGCATTCGCATCGGGCCTGGACAGACTTTCATCGCAGCACACG
AAGTGGACCATCGTATTGAAATTTTGTCACACGACAAGCGCCTGAATTCC
TGGGGTTGTAAAGGCCGCTTGGTGTGTTATACCTCGGTGGATTATAACAA
GGTGCGCTTATACGAGCACGCTGAGGCCCGTTACTCAGGAGGATCGGGGG
CACGTAAATCCGTACATATCGGTCCGGGCCAAGCCTTTTACGCTCATCAT
CACCATCACCATAAAAAGAAG

It is also possible to further isolate the epitope from detrimentally deviating the biochemical and biophysical characteristics of the resulting protein from those of the bioengineering core module that can serve as isolators, insulators, absorbers. Thus, the present invention can take into account one or more of the following parameters or factors during protein design: Flexibility/rigidity;

Rotation; Balancer—if the sequence is expanded beyond the capacity of the beta barrel, a non-antibody interacting sequence of the same size can be incorporated; Performance tags (Purification, Bioconjugation (biotin, fluorophore, coupling reagents).

A further advantage of the present invention includes the ability to use two or more Beta-barrel chains, e.g., tandem formats for increasing the number of epitopes contained within a single recombinant protein, and/or combining the epitope embedded core protein with an unmodified fluorescent protein would generate a reagent that is fluorescent to allow non-destructive detection methods. The use of these proteins allows for improved: quality control (QC), concentration, normalization. The present invention allows for the creation of a system that can be computer modeled for in silico feasibility analyses prior to synthesizing coding DNA and protein production. Further, the resulting proteins/reagents can be lyophilized/freeze-dried for stabilization.

Thus, in certain additional embodiments, the present invention include a reaction location where there is a combination of a reaction agent (binding agent) and a first chemical compound that in the presence of a molecule of interest, together with one or more reactions, results in the release of the first chemical compound to a signal detect location containing a second chemical compound capable of binding the first chemical compound and a measurement made. This describes the generation of a mobile (or non-mobile) reaction center since it describes a location where the first chemical compound and a reaction reagent, which is capable of binding the MOI, are present in a non-mobile state (ie fixed/bound). Further, it states that the presence of the MOI leads to one or more reactions that modifies the first chemical compound such that a portion is released, which can then interact with a second chemical compound at a second location and the binding of the subunit of the first compound by the second chemical compound can be measured. The reaction location can be a solid surface of a paramagnetic particle, glass, plastic, PC-board, silicon or agarose. The reaction Binding Agent is capable of binding/capturing the molecule of interest and is comprised of a bioengineered multi-epitope biomarker (ImmunoGlyph), recombinant protein/s, protein/s purified from a pathogen, whole viruses, whole bacteria, antibodies, aptamers, nanobodies or antibody mimetics.

The first chemical compound can be comprised of one, two, three, or a string of nucleic acids. The second chemical compound can be comprised of a string of nucleic acids, peptide nucleic acids or non-natural mimics of nucleic acids that have properties strictly complementary to the first chemical compound that allows for non-transient interactions between the two chemical compounds. The reaction is performed by chemical reagents that are capable of modifying the chemical properties of the first chemical compounds and occurs due to the binding of the molecule of interest. This embodiment defines the specialized chemical reagents that modify the 1st chemical compound as a condition of the presence of the MOI; specifically, these are anti-human immunoglobulin antibodies chemically crosslinked with DNA enzymes, e.g., DAM methyltransferase or other such systems. The second chemical compound can also be comprised of one, two, three or a string of nucleic acids, peptide nucleic acids or non-natural mimics of nucleic acids that have properties strictly complementary to the first chemical compound that allows for non-transient interactions between the two chemical compounds. The signal detection location comprises an area capable of performing a measurement, houses the 2nd chemical compound and that the presence of the liberated 1st chemical compound results in changes that can be detected through the measurement.

A reaction acting upon a chemical compound performed by the actions of two or more reagents that results in the chemical compound transitioning into two or more independent chemical compounds. The actions of the reagents can occur in combination. One example is a DNA methyltransferase, a type IIM restriction enzyme, and the actions of the reagents occur in sequence, e.g., when using heterodimer restriction enzymes. Thus, the actions of the reagents can be determined independently of other reactions (positive control). Alternatively, the actions of the reagents can be restricted to the presence of a MOI independently of other reactions (negative control). Moreover, the actions of the reagents to detect a MOI can be mimicked at different levels to provide a calibration of the system (calibration). In one example, one of the reagents is one-half of a heterodimer restriction enzyme. In another example, one of the reagents is the second half of a heterodimer restriction enzyme. Thus, the reaction can be divided into multiple steps that can be physically separated from each other. The reaction on the chemical compound results in the creation of two or more separate chemical compounds. The reaction can include the combined action of two or more components.

Another embodiment of the present invention includes a first protein-second protein conjugate comprising a first protein covalently linked to a second protein through a chemical crosslinker. In one non-limiting example, the first protein is an antibody, an antibody mimetic, antibody fragments and the like. In one non-limiting example, the second protein is a DNA modifying enzyme, e.g., one component of a multi-enzyme complex that modifies DNA. The chemical crosslinker can be, e.g., a heterobifunctional PEG linker, a homobifunctional linker, or any of a number of well-known chemical linkers.

Table 5 includes non-limiting examples of nucleic acid enzymes for use with the present invention.

TABLE 5
Nucleic Acid Enzymes.

| Classification | Enzyme/s | Sequence |
|---|---|---|
| “Type IIS Homodimers” | FokI | GGATG 9/13 |
| | Mva 1969I | GAATGC 1/−1 |
| | BtsI | GCAGTG 2/0 |
| “Type IIT | BbvCI | CCTCAGC −5/−2 |
| Heterodimeric” | BfiI | ACTGGG 5/4 |
| | BsrDI | GCAATG 2/0 |
| | BstNBI/BspD6I | GAGTC 4/6 |
| ”Type IIM Methylated | Dam/DpnI | GATC |
| Recognition Site” | Fsp 4H1/Bis I | ACTGGG 5/4 |
| | AluI/AluI | AGCT |

Another embodiment of the present invention includes a method to incorporate one or more short amino acid sequences within a core sequence that yields a novel protein for immunological applications. The short amino acid sequence of 4-18 amino acids represents an epitope that is recognized by an antibody. The short amino acid sequence can also represent a unique site within a protein that is desired to be targeted for antibody recognition. The amino acid sequence of an epitope can be augmented to an increased sized that confers flexibility, rigidity or expands the size to minimize structural strain on the site of insertion. The core sequence is that of a beta barrel type of protein. In one non-limiting example, the beta barrel is defined to generate fluorescence. The resulting novel protein can be placed in tandem with a fluorescent protein, or can even be combined in tandem with a separate novel protein formed by the same procedure.

FIG. 16 shows the alignment of multiple fluorescent proteins from Aequorea Victoria and Discosoma sp, Montastraea cavernosa, Zoanthus sp and Montastraea c avemosa. The overall structural elements of the eleven β-sheets are easily detected. It is also clear that the absolute amino acid sequence is not maintained over the course of evolution. The intervening sequences between the β-sheets also show divergence, which allows them to serve as insertion points for epitopes, each of which is incorporated herein by reference (SEQ ID NOS: X-Y, respectively.

Valveless hydrophobic reagent injection/removal ports.

One novel aspect of this approach is that it does not require valves due to the small port diameter, hydrophobicity of the plastic and closed piston design of the syringe pumping system. In one embodiment, the cartridge is supplied with all necessary liquids contained within the syringe pumps. Any liquids that are removed in a given step are contained within syringe pump bores avoiding contact of biohazard liquids with the operator. Initially, before use, these liquids may be contained directly within the syringe bore, on placed inside a capsule or plastic bag which is contained in the bore.

The role of the capsule would be to provide a sealed environment for the supporting electrolytes and reagents so that shelf life is increased. The encapsulation process can be any of those well-known in the testing, diagnostic and/or pharmaceutical industry. In the case where electrolyte capsulation is used, a sharp edge would be included in the syringe bore so that compressing the piston would rupture and release the contents of the capsule. Reagents which are more stable in dried form would be freeze dried on the appropriate well surface, for example 42. In FIG. 11, the cartridge 21 plugs into a connector 22, which provides electrical interconnect between the controller and cartridge.

In order for fluids to be displaced there must be a pathway for the gas contained in the fluid paths to migrate through. Because the system is closed, it ends in a sealed piston, there is no pathway for gas or fluid to escape. Also, because the diameter of the pathway is small and the surface of the pathway is hydrophobic, force must be applied to form a new liquid surface. This force must overcome the surface tension of the liquid and lack of wettability of the plastic body which increases the surface energy. The syringe or piston pumping force is great enough to overcome this, but gravity is not. This approach, which avoids the need for valves, reduces the manufacturing cost of the cartridge.

Sample Injection port. The cartridge is designed as a sealed system and the injection port allows samples to be introduced but prevents contained liquids from escaping by gravity. This aids in preventing operators from being exposed to harmful pathogens or biohazards contained in the patient sample. The injection port includes an elastomer 44 with a small hole in it. The elasticity allows a larger diameter sample injection instrument such as a capillary, needle, or pipette to pass through the smaller hole and also closes the hole upon removal of the sample injector. The combination of the hydrophobicity of the elastomer and small hole size prevent liquid escape by gravitational forces, again utilizing the principle of an energy barrier provided by surface tension and hydrophobicity.

Syringe pumps can be external to, and/or be incorporated in, the cartridge body.

Planar reaction center embodiment. FIG. 4 shows the front-end chemistry and substrate of the present invention that includes an “immunoglyph” 2, which is tied to, e.g., micrometer sized magnetic beads or a planar non-magnetic substrate 1, but other substrates are possible.

Thin layer cell with fixed (planar non-magnetic) reaction domains. When the planar front-end substrate is used magnets are not required, but may also be used. The well volume is reduced in order to facilitate mass transport of the signal molecules to the capture molecule functionalized electrodes. In a particularly elegant embodiment, the planar front-end substrate is placed on the face opposite of the array and the dimension between them is minimized forming a thin layer cell. This increases the sensitivity of the signal response and therefore reduces measurement time by increasing the signal concentration due to volume reduction and mass transport due to increased concentration and reduced diffusion length between the source Mehrdraht Dortmund Oberfläche (MDO) and electrode planes.

Cartridge Orientation. FIGS. 12A to 12C show a cartridge embodiment with a vertical orientation. This is beneficial for avoiding or limiting false positive signals that occur due to uncaptured front-end substrate particles. In a vertical orientation these particles often fall to the well bottom, which is benign in a vertical orientation, but could result in false positive signals in a horizontal configuration where the array electrodes are on the bottom of the cell. Other orientations are possible, for example, a horizontal configuration with the array on top would also reduce the possibility of false positives.

Array Metallization stack. The metal stack starts with a glue layer 48, which has to be reactive in order to adhere to the substrate, for example titanium, chrome, or tantalum. High reactivity is important because any glue layer metal which is exposed to the electrolyte for example by diffusing through the stack, forms a highly stable oxide. A highly stable oxide will not be electrochemically active in the potential ranged used to detect the signal molecule redox label. The remaining metals are noble, that is, they do not react with oxygen and their redox potentials are positive of the potential range used for signal molecule redox label detection. Examples are gold, platinum, palladium, iridium, and rhodium. When titanium is used as a glue layer, there is usually an intermediate layer 49 used to facilitate adhesion in harsh environments, for example palladium or platinum. Typical currents are small, in the nanoamp range, therefore the metal path resistance can be as high as several megaohms without significantly affecting the measurement. This allows noble metal stack thicknesses as low as 100-2,000 angstroms, which reduces cost.

Chemical constituents and recipe steps (protocol) that insure that only positive cases result in a signal Note that two enzymes, one for methylation, and the other for cleavage of the methylated target are required for cleavage. Cleavage cannot occur if methylation has not taken place. One of the enzymes is conjugated to the secondary antibody, this can be either the methylation or the restriction (cleavage) enzyme. The other enzyme is free in solution. These enzymes are added in different steps, separated by rinses. In order for both enzymes to be present together, one must be captured via the binding of the secondary to the primary antibody, otherwise it will be removed by the intermediate rinse. This scheme and sequence assures that only captured pathogens or antibody targets cleave to generate a signal. Other schemes can also be devised where cleavage requires two distinct chemical alterations, each carried out by different chemical constituents, where one is conjugated to an element which is captured in the presence of the target and the other free to move about in solution, can be devised and are covered as an alternate embodiment.

Table 6 includes non-limiting examples of epitopes for use with the present invention that can be inserted into the beta-barrel proteins of the present invention using conventional methods, and have SEQ ID NOS: Y-Z.

| | | Epitopes | | | | | |
|---|---|---|---|---|---|---|---|
| | | (SEQ ID | | | | | |
| Pathogen | Type | NOS) | | | | | |
| Epstein- | Virus | GGWYSFD | YTDSSM | DNYWSF | ELISSCLV | AGAGGG | |
| Barr | | SPYLMSIT | AVTLMK | SDSTYW | WSARGCL | AGGAGA | |
| | | EMRLR (36) | FASNFLF | TLRYSSG | FGGGI | GGGAGG | |
| | | | (62) | (85) | (104) | AGC (120) | |
| Hepatitis | Virus | FWRGDLV | TVSTEQ | LEVGKQ | “EVLPPPR | GLLRYHT | FWRGDLV |
| A | | FDFQV (37) | NVPDPQ | RLKYAQ | KKKGLFS | YARFG | FDFQV |
| | | | VGI (63) | EEL (86 | Q (105)” | (121) | (VP3)(132) |
| | | FNASDSVG | SHSQLGP | SHSVTKS | | | |
| | | QQIKVIP | PVGPP | LRVFGGP | | | |
| | | (38) | (64) | P (87) | | | |
| Hepatitis B | Virus | CWGELMN | CLTFGRE | TPPAYR | PPNAPILS | EALESPE | FGRETVL |
| | | LATWVGS | TVLEY | (88) | TLPE (106) | H (HBeAg | EYLVSFG |
| | | NLEDPASR | (65) | | | 49- | (HBeAg |
| | | E (39) | | | | 56)(122) | 120-133) |
| | | | | | | | (133) |
| | | ASRDLVV | | | | | |
| | | NY (40) | | | | | |
| Hepatitis C | Virus | ANLINEFD | REVLYR | KTKRNT | RKTSERS | QKKNKR | PPLLESW |
| | | DLAS (41) | EFDEME | NRRPQD | QPRGRRQ | NTNRRPQ | KDPDYVP |
| | | | (66) | VKF (89) | P (107) | DV (123) | PWHG |
| | | | | | | | (N55)(134) |
| | | GGVYLLPR | ETWKKP | AFASRG | | | |
| | | RGPRLGV | DYEPPV | NHVSPT | | | |
| | | WHPRGSR | VHG | HYVPES | | | |
| | | PSWGPTDP | (NS5 | DA (NS4) | | | |
| | | RRWH″NH | 2299- | (90) | | | |
| | | VSPTHYVP | 2313) (67) | | | | |
| | | ESDAAAR | | | | | |
| | | (NS4 1927- | | | | | |
| | | 1943)(42) | | | | | |
| Leishmaina | Parasite | FFTEEFKR | RRVAVL | | | | |
| (Visceral) | | KNKGKNL | VLLDRL | | | | |
| | | ASSHR (43) | (68) | | | | |
| | | RFFVQGD | | | | | |
| | | GIGQHSLQ | | | | | |
| | | EALERR | | | | | |
| | | (44) | | | | | |
| Malaria | Parasite | SASDQPTQ | NEIELSA | SVPTNLD | SVPTNLD | IVEVEEIL | KNEEFLN |
| | | YEEEMTD | RDVLENI | YVPQFLR | YVPQYLR | PE (124) | DRCDIC |
| | | YQK (45) | G (69) | W (91) | W (108) | | (135) |
| | | VKEKEEV | KSTYLTE | PEPTVTN | GFDDGSA | DEMLDPE | DPNANPN |
| | | KEKEE (46) | PILTEEH | EEY (92) | FGGGLPF | ASF(125) | V (136) |
| | | | L (70) | | (109) | | |
| T.cruzi | Parasite | FAELLEQQ | KAAAAP | KAAIAPA | GDKPSPF | KQKAAE | PVVAES |
| | | KNAQFPG | A (TcE) | (TcE)(93) | GQAAAA | ATK | (202- |
| | | KASK | (71) | | DK (Pep- | (CRA) | 207)(137) |
| | | (KMP11)(47) | | | 2)(110) | (126) | |
| | | AEPKPAEP | AEPKSA | GTSEEGS | SPFGQAA | | VETLL |
| | | KS (TcD- | EPKP | RGGSSM | AGDK | | (62-66 |
| | | 1/Ag13) (48) | (TcD-2) | PS (TcLo | (B13)(111) | | OspC (138) |
| | | | (72) | 1.2) (94) | | | |
| | | PPSGTENK | DSSAHST | FGQAAA | CNNSGKD | | |
| | | PAT ((49)) | PSTPA | GDKPSL | GN (OspC | | |
| | | | (SAPA)(73) | (95) | 19-27) | | |
| | | | | | (112) | | |
| Lyme | Bacterial | KIEFSKFT | MKKND | DTGSERS | INKLEAK | CVQEGV | AKKAILIT |
| Disease | | VKIKNKD | QIVAAIA | IRYRRRV | KTSLKTY | QQEGAQ | DAAKDK |
| (Borrelia | | (Erp)(50) | LRGVA | Y | SEYEEQ | QP (pFlaB) | G (Osp |
| burgdorferi) | | | (74) | (pp35)(96) | (OspF)(113) | (127) | Type K) |
| | | | | | | | (139) |
| | | MTLFLFIS | LVACSIG | SQVADK | KDDPTNK | SDISSTTG | |
| | | CNNSGKD | LVERTN | DDPTNK | FYQSVIQ | KPDSTG | |
| | | GNTSAB)IP | AA | FY (VlsE | (VlsE | (VlsE 96) | |
| | | SKENAKLI | (DbpB) | 21)(97) | 26)(114) | (128) | |
| | | VYFYDNV | (75) | | | | |
| | | YAG (pLA7 | | | | | |
| | | 91-110) (51) | | | | | |
| | | LRKVGDS | AESPKKP | PVVAESP | VQEGVQ | | |
| | | VKAASKE | (76) | KKP | QEGAQQP | | |
| | | (VlsE 3326) | | (Outer | (115) | | |
| | | (52) | | Surface | | | |
| | | | | protien | | | |
| | | | | C)(98) | | | |
| Syphilis | Bacterial | ASGAKEE | VMYASS | DYARVM | EAAFREL | | |
| (Treponema | | AEKKAAE | G (77) | Y (99) | (116) | | |
| pallidum) | | QRALL (53) | | | | | |
| Tick borne | KFDWN | TSGKDIVQ | KDGKSW | ETKAWY | LEIGYERF | TTNRFAK | |
| Fever (A. | TPDP | FAKAVEIS | KLESHK | PYLKDG | KTKGIRD | T(129) | |
| phagocytophilum) | | (54) | FDWNTP | KSWKLE | SGSKE | | |
| haBacterial | | | DPRIGFK | SHKFDW | (117) | | |
| | | | D (78) | NTPD | | | |
| | | | | (100) | | | |
| Chlamydia | Fungal | NQSTVKT | VLKTDV | TKDASID | TRLIDER | TLNPTIA | SANNDAE |
| (Chlamydia | | (55) | NKE (79) | YHE (101) | AAH (118) | G (130) | IGNLI |
| trachomitis) | | | | | | | (140) |
| | | VLAEAIGL | SANNDA | GDBRKA | SAPLKQI | NAGKEG | GAIIFQQV |
| | | R(56) | EIGNLI | MFEDIA | AANAG | AIIFQQ | MSRS |
| | | | (80) | (102) | (119) | (131) | (141) |
| Lupus/Sjpus/ | Autoimmune | LQPFPQPE | | | | | |
| SjV | | LPYPQPQ | | | | | |
| syndromw | | (58) | | | | | |
| Prostein | Cancer | GPKPGAPF | | | | | |
| | | P (59) | | | | | |
| Cat | Allergen | AQYKALP | DAKMTE | | | | |
| | | VVLENA | EDKENA | | | | |
| | | (60) | LS (82) | | | | |
| | | | EICPAVK | | | | |
| | | | RDVDLF | | | | |
| | | | LTGT | | | | |
| | | | (83) | | | | |
| White Birch | Allergen | TLLRAVES | EVDHTN | | | | |
| | | YLLA (61) | FKYNYS | | | | |
| | | | (84) | | | | |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

1. A device comprising:
one or more wells on a substrate onto which one or more molecules of interest (MOI) binding reagents are attached, wherein each of the one or more MOI binding reagents is within a molecular proximity of one or more detectable signal molecules, wherein each of the one or more detectable signal molecules comprise one or more signal molecules that are releasable in the presence of the MOI by one or more enzymes and the signal is detected by an electronic detection system.

2. The device of claim 1, wherein a release of the one or more signal molecules is measured electronically to determine a quantity of the one or more MOIs in the sample.

3. The device of claim 1, wherein the device comprises at least one of:
a cartridge that comprises the substrate, the wells, and the MOI, in fluid communication with one or more ports 34-36 that facilitate the transfer of chemical reagents into and out of the wells;
a pump in fluid communication with the ports, which the pump is external to the cartridge;
the cartridge comprises a body constructed of plastic, and wherein the cartridge is machining, injection molding, or 3D printing;
the device does not require valves;
a diameter of one or more fluid pathways is hydrophobic;
the cartridge includes one or more circuits in electrical communication with the substrate and the wells, wherein the one or more circuit convert electrochemical measurements into electrical signals; and
an electronic signal detected is at least one of: non-transient, cumulative, or coded.

4. (canceled)

5. (canceled)

6. (canceled)

7. (canceled)

8. (canceled)

9. (canceled)

10. The method of claim 1, wherein at least one of: the MOI is selected from pathogen-specific antibodies, auto-antibodies, viruses, bacteria, parasites, fungi, helminths, chemicals, illicit drugs, drugs, toxins, hormones, proteins, lipids, glycogens, carbohydrates, biological therapies, pathogen biomarkers, cardiac biomarkers, disease biomarkers, or cancer biomarkers;
the MOI binding reagent comprising one or more polypeptides that specifically interact with the MOI, antibodies, an antibody fragment, Fab, Fab', Fab'-SH, F(ab')₂, Fv, or scFv fragment, nanobody, a bi-specific antibody, pathogen lysate, recombinant protein, chimeric proteins peptide, glycopeptide, lectin or carbohydrate;
the one or more MOI binding reagents, one or more detectable signal, and one or more enzymatic reactions, are incorporated into a cartridge in fluid communication with an electronic surface capable of detecting the one or more signal molecules and the one or more validation signals.

11. The method of claim 1, wherein the samples are selected from at least one of biological fluids, water, air and surfaces.

12. (canceled)

13. (canceled)

14. The method of claim 1, wherein a signal measured electronically are solid state detectors.

15. The method of claim 1, wherein the enzymatic reaction occurs in one or more closed system cartridges.

16. The method of claim 1, wherein the electronically measured signal are an electrochemical, a surface plasmon resonance, an infrared, a capacitance coupled, a dye-coupled fiber optic, a hyperspectral sensor or a cantilever sensor.

17. The method of claim 1, further comprising an internal control that is at least one of: an internal calibration for signal intensity, signal production kinetics, or signal position.

18. The method of claim 1, wherein at least one of:
the electronic detection system comprises multiple independent electrodes that allows a subset to include: an internal calibration for signal intensity, signal production kinetics, and a signal position;
the electronic detection system comprises a metal stack and a reactive glue layer that reacts with titanium, chrome, or tantalum, wherein the reactive glue layer is at least one of titanium, palladium or platinum;
the electronic detection system comprises a glue layer-metal stack that is exposed to one or more electrolyte in solution;
the electronic detection system comprises a stable oxide that is not electrochemically active in the potential ranged used to detect the signal molecule;
the electronic detection system comprises a metal layer that compromises noble metals that do not react with oxygen and their redox potentials are positive in the range used for signal molecule redox label detection, wherein the noble metal is selected from at least one of gold, platinum, palladium, iridium, or rhodium;
the electronic detection system detects currents the nanoamp range, and a metal path resistance can be as high as several megaohms without significantly affecting the measurement or the electronic detection system comprises a noble metal stack with a thicknesses of 100-2,000 angstroms.

19. (canceled)

20. (canceled)

21. (canceled)

22. (canceled)

23. (canceled)

24. (canceled)

25. (canceled)

26. (canceled)

27. A valveless device for detecting a molecule of interest (MOI) comprising:
one or more reagents in a liquid;
a pump in fluid communication with the reagents and a reaction chamber, wherein the reaction chamber comprises one or more substrates onto which one or more MOI binding reagents are attached, wherein each of the one or more MOI binding reagents is within a molecular proximity of one or more detectable signal molecules;
a port for introducing a sample suspected of comprising the MOI into the reaction chamber;
wherein each of the one or more detectable signal molecules comprise one or more signal molecules that are releasable in the presence of the MOI by one or more enzymes; and
wherein a release of the one or more signal molecules is measured to determine a quantity of the one or more MOIs in the sample; and
one or more sensors capable of detecting the one or more signal molecules released if the MOI is present in the sample.

28. The device of claim 27, wherein at least one of: the liquid and pump are contained within a cartridge:
the liquid, pump, port, reaction chamber, and sensors are sealed within a closed system
the one or more reagents are contained within a disposable bag or capsule;
the port comprises an elastomeric seal that prevent liquid from escaping the one or more conduits within the device;
the substrate is a planar surface; or
the one or more sensors are defined further as capture molecule functionalized electrodes.

29. (canceled)

30. (canceled)

31. (canceled)

32. (canceled)

33. (canceled)

34. The device of claim 27, wherein the cartridge includes one or more circuits in electrical communication with the substrate and the wells, wherein the one or more circuit convert electrochemical measurements into electrical signals.

35. The device of claim 27, wherein an electronic signal detected is at least one of:
non-transient, cumulative, or coded.

36. The method of claim 27, wherein at least one of:
the MOI is selected from pathogen-specific antibodies, auto-antibodies, viruses, bacteria, parasites, fungi, helminths, chemicals, illicit drugs, drugs, toxins, hormones, proteins, lipids, glycogens, carbohydrates, biological therapies, pathogen biomarkers, cardiac biomarkers, disease biomarkers, or cancer biomarkers;
MOI binding reagent comprising one or more polypeptides that specifically interact with the MOI, antibodies, an antibody fragment, Fab, Fab', Fab'-SH, F(ab')₂, Fv, or scFv fragment, nanobody, a bi-specific antibody, pathogen lysate, recombinant protein, chimeric proteins peptide, glycopeptide, lectin or carbohydrate; or
the one or more MOI binding reagents, one or more detectable signal, and one or more enzymatic reactions, are incorporated into a cartridge in fluid communication with an electronic surface capable of detecting the one or more signal molecules and the one or more validation signals.

37. The method of claim 27, wherein the samples are selected from at least one of biological fluids, water, air and surfaces.

38. (canceled)

39. (canceled)

40. The method of claim 27, wherein a signal measured electronically are solid state detectors.

41. The method of claim 27, wherein the enzymatic reaction occurs in one or more closed system cartridges.

42. (canceled)

43. The method of claim 27, further comprising an internal control that is at least one of: an internal calibration for signal intensity, signal production kinetics, or signal position.

44. The method of claim 27, wherein at least one of:
the one or more sensors comprises multiple independent electrodes that allows a subset to include: an internal calibration for signal intensity, signal production kinetics, and a signal position;
the one or more sensors comprises a metal stack and a reactive glue layer that reacts with titanium, chrome, or tantalum;
the one or more sensors comprises a stable oxide that is not electrochemically active in the potential ranged used to detect the signal molecule;
the one or more sensors comprises a glue layer-metal stack that is exposed to one or more electrolyte in solution;
the one or more sensors comprises a metal layer that compromises noble metals that do not react with oxygen and their redox potentials are positive in the range used for signal molecule redox label detection;
the one or more sensors detects currents the nanoamp range, and a metal path resistance can be as high as several megaohms without significantly affecting the measurement;
the one or more sensors comprises a noble metal stack with a thicknesses of 100-2,000 angstroms; or
the one or more sensors are an electrochemical, a surface plasmon resonance, an infrared, a capacitance coupled, a dye-coupled fiber optic, a hyperspectral sensor or a cantilever sensor.

45. (canceled)

46. (canceled)

47. (canceled)

48. (canceled)

49. (canceled)

50. (canceled)

51. (canceled)

52. (canceled)
